SEQUENCE LISTING (34 sequences)

SEQ ID NO: 1 - protein, 1134 aa, Homo sapiens
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKPGIPAAEVGIGVVAEADAADAAGFPVPPDMEDDYEPELLLMPSNQPVNQPILAAAQSLHREATKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 2 - protein, 1066 aa, Homo sapiens
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 3 - protein, 222 aa, Homo sapiens
MPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKVRVLSGEISKIPNSPWLGVLIGTCLILYLVIFVA

SEQ ID NO: 4 - protein, 1066 aa, Mus musculus
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPNASPGDAGEQAIRQILDEAGKVGELCAGKERREILGTCKMLGQMTDQVADLRARGQGASPVAMQKAQQVSQGLDVLTAKVENAARKLEAMTNSKQSIAKKIDAAQNWLADPNGGPEGEEQIRGALAEARKIAELCDDPKERDDILRSLGEIAALTSKLGDLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKAQMQEAMTQEVSDVFSDTTTPIKLLAVAATAPPDAPNREEVFDERAANFENHSGRLGATAEKAAAVGTANKSTVEGIQASVKTARELTPQVISAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 5 - RNA, 21 nt, artificial
  synthetic oligonucleotide
cuggcuugcagauccaaauuu

SEQ ID NO: 6 - RNA, 21 nt, artificial
  synthetic oligonucleotide
uucuccgaacgugucacgunn

SEQ ID NO: 7 - DNA, 20 bp, artificial
  synthetic oligonucleotide
agtgggagaggtgcaggaaa

SEQ ID NO: 8 - DNA, 23 bp, artificial
  synthetic oligonucleotide
tgaaacacactgtccctctttga

SEQ ID NO: 9 - DNA, 22 bp, artificial
  synthetic oligonucleotide
cttcacgcctgtgtgcttttat

SEQ ID NO: 10 - DNA, 18 bp, artificial
  synthetic oligonucleotide
agggtgcaggacgttcca

SEQ ID NO: 11 - DNA, 20 bp, artificial
  synthetic oligonucleotide
tgggacaacttgcaaacctg

SEQ ID NO: 12 - DNA, 21 bp, artificial
  synthetic oligonucleotide
ccagagtaggtctgttttcaa

SEQ ID NO: 13 - DNA, 26 bp, artificial
  synthetic oligonucleotide
aacaggcattattgtctttgaaaaag

SEQ ID NO: 14 - DNA, 26 bp, artificial
  synthetic oligonucleotide
tctcattctgtggctgtgtactctct

SEQ ID NO: 15 - DNA, 18 bp, artificial
  synthetic oligonucleotide
cctggagggcttggagat

SEQ ID NO: 16 - DNA, 19 bp, artificial
  synthetic oligonucleotide
atcctacggctggctgtga

SEQ ID NO: 17 - DNA, 28 bp, artificial
  synthetic oligonucleotide
gttttccatcttttccagttgtctataa

SEQ ID NO: 18 - DNA, 22 bp, artificial
  synthetic oligonucleotide
catatggcctgtgaagctttca

SEQ ID NO: 19 - DNA, 24 bp, artificial
  synthetic oligonucleotide
cgcagagttatcgtgccagcagat

SEQ ID NO: 20 - DNA, 24 bp, artificial
  synthetic oligonucleotide
ccatattctttcaccgcccactcc

SEQ ID NO: 21 - DNA, 25 bp, artificial
  synthetic oligonucleotide
gtgtcactatggagctctcacatgt

SEQ ID NO: 22 - DNA, 20 bp, artificial
  synthetic oligonucleotide
gtttcccttcagcggctctt

SEQ ID NO: 23 - DNA, 18 bp, artificial
  synthetic oligonucleotide
tgtgtgctggacgctgga

SEQ ID NO: 24 - DNA, 19 bp, artificial
  synthetic oligonucleotide
cactgccccatgacgtgat

SEQ ID NO: 25 - DNA, 20 bp, artificial
  synthetic oligonucleotide
ggctggcagtctccctaaaa

SEQ ID NO: 26 - DNA, 25 bp, artificial
  synthetic oligonucleotide
atcaaggagctcaatctcaaaaaag

SEQ ID NO: 27 - DNA, 25 bp, artificial
  synthetic oligonucleotide
caggcagctatatgaagaggagatc

SEQ ID NO: 28 - DNA, 21 bp, artificial
  synthetic oligonucleotide
atggacagcaccacagatgtg

SEQ ID NO: 29 - DNA, 24 bp, artificial
  synthetic oligonucleotide
gcgaggactttaatcttggtgatg

SEQ ID NO: 30 - DNA, 21 bp, artificial
  synthetic oligonucleotide
tggtcttttggatggtttgca

SEQ ID NO: 31 - DNA, 17 bp, artificial
  synthetic oligonucleotide
cctcgtccgggttggaa

SEQ ID NO: 32 - DNA, 24 bp, artificial
  synthetic oligonucleotide
taaatgctggtggcatatctctct

SEQ ID NO: 33 - DNA, 20 bp, artificial
  synthetic oligonucleotide
ggcctccaaggagtaagacc

SEQ ID NO: 34 - DNA, 20 bp, artificial
  synthetic oligonucleotide
aggggagattcagtgtggtg

(19) United States
(12) Patent Application Publication
(10) Pub. No.: US 2014/0107176 A1
(43) Pub. Date: Apr. 17, 2014

(54) METHOD OF MODULATING A PROSTATE CANCER CELL

(71) Applicant: Agency for Science, Technology and Research, Singapore (SG)

(72) Inventors: Edwin Chong-Wing Cheung, Singapore (SG); Kern Rei Chng, Singapore (SG)

(73) Assignee: Agency for Science, Technology and Research

(21) Appl. No.: 13/651,141

(22) Filed: Oct. 12, 2012

(51) Int. Cl. G01N 33/68

(52) U.S. Cl. CPC G01N 33/6893
USPC 514/44 A; 435/375; 435/6.14

(57) ABSTRACT

There is provided a method of modulating a prostate cancer cell, the method comprising administering to the prostate cancer cell an agent that augments vinculin expression levels in the cell.

### FIELD OF THE INVENTION

The present invention relates generally to methods of modulating a prostate cancer cell, including invasiveness, and to methods of diagnosing and treating prostate cancer.

### BACKGROUND OF THE INVENTION

The androgen receptor (AR) occupies a central role in the biology of both normal prostate development and prostate cancer progression (1). AR is a member of the nuclear hormone receptor superfamily that directs the transcriptional regulation of genes governing a wide variety of cellular processes including cell cycle, cell proliferation, survival, and differentiation (1). Upon activation by androgens, AR will dissociate from heat shock proteins, dimerize, and translocate from the cytoplasm into the nucleus where it recognizes and binds to androgen response elements near target genes (1). In normal cells the transcriptional activity of AR is delicately controlled by the coordinated recruitment of specific coregulatory proteins (i.e. coactivators, corepressors, collaborative factors, etc). However, these coregulatory proteins may become aberrantly expressed in prostate cancers, resulting in a deregulated AR transcriptional network (2, 3).

Collaborative factors of AR that are frequently over-expressed in prostate cancers include FoxA1, GATA2, and members of the ETS family (4, 5). The ETS family has received much attention lately because recent studies by Chinnaiyan and colleagues revealed that the majority of prostate cancers harbor recurrent fusion transcripts between the promoter region of the AR direct target gene, TMPRSS2, with different ETS members, resulting in the androgen stimulation and over-expression of ETS transcription factors (5, 6).

The most important and common ETS fusion transcription factor appears to be TMPRSS2:ERG, which is detected in approximately half of all analyzed localized prostate cancers (6). Intriguingly, in contrast to its counterparts ETS1 and ETV1 (7, 8), ERG was shown to attenuate AR-dependent transcription (9, 10). This finding suggests that the induction of ERG by AR could possibly feedback to perturb and deregulate the transcriptional network of AR. The detailed mechanism of attenuation and functional consequences associated with AR and ERG transcriptional crosstalk are currently unclear.

The collaboration between AR and ERG is of exceptional therapeutic interest as it represents a cancer specific transcription co-operation that does not exist under normal circumstances. Even though both factors are crucial in prostate cancer progression, their relationship with transcriptional corepressors remains largely unknown.

### SUMMARY OF THE INVENTION

In one aspect, the invention provides a method of modulating a prostate cancer cell, the method comprising administering to the prostate cancer cell an agent that augments vinculin expression levels in the cell.

The prostate cancer cell may be a prostate cancer cell that has been identified as having low expression levels of vinculin as compared with a non-cancerous prostate cell. The prostate cancer cell may be an in vitro cell or may be an in vivo cell.

The agent may be an expression vector encoding vinculin, including encoding a vinculin protein comprising a sequence of any one of SEQ ID NOs.: 1 to 4.

The agent may an agent that inhibits the activity of one or more of ERG, EZH2, HDAC1, HDAC2 and HDAC3, including a chemical inhibitor, an siRNA, an antisense RNA molecule or a DNA enzyme. The agent may comprise multiple agents. Each of the multiple agents inhibits one of the corepressor proteins ERG, EZH2, HDAC1, HDAC2 and HDAC3.

In another aspect, the invention provides a method of diagnosing prostate cancer disease state in a subject, the method comprising detecting vinculin levels in a sample of prostate cells obtained from a subject and comparing the levels with vinculin levels in a non-cancerous prostate cell, wherein vinculin expression levels that are lower in the sample obtained from the subject as compared to vinculin expression levels in a non-cancerous prostate cell are indicative of prostate cancer.

Lower vinculin expression levels in the prostate cancer cell may be indicative that the prostate cancer cell from the subject has a predisposition to be an invasive cancer.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the figures, which illustrate, by way of example only, embodiments of the present invention:

FIG. 1. Androgen stimulates distinct expression and chromatin binding profiles for AR and ERG in prostate cancer cells. Time course expression analysis of AR and ERG (A) mRNA and (B) protein levels in VCaP cells after stimulation with 10 nM DHT at various time points. Error bars represent S.E.M of at least 3 independent repeats. (C) Androgen stimulates AR and ERG binding to chromatin. Hormone depleted VCaP cells were treated with 100 nM DHT for 0, 2, or 18 hrs. Cells were cross-linked with formaldehyde and chromatin was immunoprecipitated with antibodies against AR or ERG. Immunoprecipitated DNA was quantified with qPCR for specific binding sites and a randomly selected genomic location as control (ctrl) region. Error bars represent S.E.M of at least 3 independent experiments.

FIG. 2. Global analysis of AR and ERG binding across the prostate cancer genome. (A) Venn diagram illustrating the overlap of AR and ERG cistromes in VCaP cells treated with 100 nM DHT at various time points (0, 2, 18 hrs). (B) Weblogo output of top AR (left) and ERG (right) enriched motifs from ChIP-Seq peaks. A de novo motif discovery algorithm, MEME, was performed on the top 1000 ranked AR and ERG (DHT) ChIP-Seq peaks (+/−50 bp from center of the ChIP-Seq peak). (C) Heatmap representation of sorted ChIP-Seq signals of AR and ERG binding events in VCaP cells. Signals are centralized to either the center of AR or ERG ChIP-Seq peak (−/+2 kb). Corresponding occurrence of predicted ARE and ETS binding motif are depicted in heatmap for on the right. (D) A comparison of the average AR and ERG ChIP-Seq tag intensities at different subsets of the AR and ERG cistrome after 0, 2, and 18 hrs of androgen stimulation. (E) Global distribution of AR and ERG binding events with respect to the transcription start sites (TSSs) of RefSeq genes. (F) Conservation analysis of AR and ERG binding sites. Comparison of the average binding intensities of (G) AR at AR binding sites with or without ERG occupancy, and (H) ERG at ERG binding sites with or without AR occupancy.

FIG. 3. ERG attenuates androgen-dependent transcription by inhibiting AR binding. Snapshots showing the co-localization of AR and ERG binding sites at two model AR target genes: (A) KLK3 and (B) FKBP5. The black arrows indicate the co-localized AR and ERG binding sites examined. (C) Western blot analysis showing AR and ERG expression in androgen deprived VCaP cells treated with EtOH or 10 nM DHT for 18 hrs after being transfected with control siRNA or siRNA against AR or ERG. GAPDH was used as a loading control. (D) and (E) ERG regulates AR-dependent transcription. VCaP cells were transfected with control siRNA or siRNA targeting AR or ERG. After 8 hrs of EtOH or 10 nM DHT stimulation, cells were harvested for total RNA, converted to cDNA before quantifying gene expression levels. GAPDH was used as a control for internal normalization. Error bars represent S.E.M of at least 3 independent experiments. (F) and (G) ERG inhibits AR binding. VCaP cells transfected with control siRNA or siRNA targeting ERG were deprived of androgens for 24 hours before being stimulated with EtOH or 100 nM DHT for 2 hrs. ChIP assays with antibodies against AR were performed and immunoprecipitated DNA was quantified with qPCR for specific binding sites. Error bars represent S.E.M of at least 3 independent experiments.

FIG. 4. HDACs and EZH2 are recruited to AR binding sites. (A) Boxplots showing the relative mRNA expression levels of HDAC1, HDAC2, HDAC3, and EZH2 in clinical prostate samples from the Yu et al (49) study, which has been deposited in the Oncomine database. (B) Androgen-depleted VCaP cells were treated with either EtOH or 100 nM DHT for 2 hrs. The cells were then double crosslinked with DSG followed by formaldehyde. Chromatin was immunoprecipitated with antibodies against HDAC1, HDAC2, HDAC3, or EZH2 Immunoprecipitated DNA was quantified with qPCR for specific binding sites and a randomly selected genomic location as a control (ctrl) region. Error bars represent S.E.M of at least 3 independent experiments.

FIG. 5. An integrated transcriptional network of AR, ERG, HDACs and EZH2 in prostate cancer. (A) Distribution of predicted AR and ETS binding motifs at HDAC1, HDAC2, HDAC3, and EZH2 ChIP-Seq binding sites. (B) Bar chart showing the percentage binding sites located at the promoter proximal (−/+3 kb from TSS) and distal regions for AR, ERG, HDAC1, HDAC2, HDAC3, and EZH2. (C) Heatmap representation of sorted ChIP-Seq signals of AR, ERG, HDAC1, HDAC2, HDAC3, and EZH2 binding events in VCaP cells. Signals are centralized to either the center of AR or ERG ChIP-Seq peak (−/+2 kb). (D) A comparison of the average ChIP-Seq tag intensities of AR, ERG, HDAC1, HDAC2, HDAC3, and EZH2 at different subsets of the AR and ERG cistrome after 2 hrs of androgen stimulation.

FIG. 6. Co-recruitment of ERG, HDACs, and EZH2 to ARBS attenuates AR-dependent transcription. (A) Snapshots showing the localization of HDAC1, HDAC2, HDAC3, and EZH2 with AR and ERG at model AR target genes, PSA and FKBP5. (B) Endogenous interactions between AR and ERG with HDAC1, HDAC2, HDAC3, and EZH2 in VCaP cells were detected by co-immunopreciptation. (C) VCaP cells grown in full serum (top) or depleted with hormones and treated with 10 nM DHT (bottom) were subjected to varying concentrations of TSA for 24 hours. Total RNA from the treated cells were then harvested and converted to cDNA before quantifying for gene expression levels by qPCR. GAPDH was used as an internal normalization control. Error bars represent S.E.M of at least 3 independent experiments. (D) VCaP cells grown in full serum (top) or depleted with hormone and treated with 10 nM DHT to 8 hours (bottom) were subjected to 3 μM DZNep treatment for 24 (top) or 48 (bottom) hrs. Total RNA was extracted and processed as described in FIG. 6C. Error bars represent S.E.M of at least 3 independent experiments.

FIG. 7. ERG, HDACs, and EZH2 promote cell invasiveness and inhibit epithelial differentiation by antagonizing AR-dependent transcription of cell adhesion molecules. (A) A network showing prostate cancer clinical gene signatures that are related to ERG-associated androgen upregulated genes. Oncomine Molecular Concept Map analysis was performed to compare ERG-associated (5 kb from TSS) androgen induced genes (>2 fold) that were identified in our study against clinical prostate cancer gene signatures available in the Oncomine database. The criteria for significant associations between node is defined as OD>=2; p-value <le-4. (B) Boxplot showing the relative mRNA expression of VCL in clinical prostate samples from the Yu et al (49) study, which has been deposited in the Oncomine database. (C) Scatterplot showing the relative mRNA expression of VCL and its corresponding ERG mRNA expression in clinical prostate samples from the Yu et al (49) study. (D) Kaplan-Meier survival curve (using data from the MSKCC dataset) showing the differences in the risk of biochemical relapse between prostate cancer patients expressing high (red line) or low (green line) VCL levels. (E) Snapshot showing the localization of AR, ERG, HDAC1, HDAC2, HDAC3, and EZH2 at the regulatory region of VCL. (F) Western blot analysis showing VCL expression in VCaP cells growing in normal full serum media treated with control siRNA or siRNA against VCL. GAPDH was used as a loading control. (G) The effect of AR and ERG silencing on VCL gene expression was analyzed as described in FIG. 3D-E. Error bars represent S.E.M of at least 3 independent experiments. (H) The effect of TSA on the androgen-dependent gene regulation of VCL was analyzed as described in FIG. 6C. Error bars represent S.E.M of at least 3 independent experiments. (I) The effect of DZNep on the androgen-dependent gene regulation of VCL was analyzed as described in FIG. 6D. Error bars represent S.E.M of at least 3 independent experiments. (J) VCaP cells transfected with control siRNA or siRNA targeting VCL were subjected to Matrigel invasion assay. The number of cells per high power field (HPF) that passed through the transwell was counted. Error bars represent S.E.M of at least 3 independent experiments. (K) The effect of VCL suppression on cancer cell survival was assessed by flow cytometry. siRNA transfected cells were fixed prior staining with propidium iodide for flow analysis. Analysis was presented as % of total number of gated cells in the subG1 phase. (L) The effect of VCL suppression on cancer cell proliferation was assessed by flow cytometry. siRNA transfected cells were incubated with BrdU for 48 hours prior to fixing. The fixed cells were then stained with BrdU antibodies and 7-AAD before flow cytometry analysis was performed. Analysis was presented as % of total number of gated cells in the S phase of the cell cycle.

FIG. 8. A table listing ChIP-qPCR primers used in the Examples set out below. The sequences correspond to SEQ ID NOs.: 7-18, respectively.

FIG. 9. A table listing cDNA RT-qPCR primers used in the Examples set out below. The sequences correspond to SEQ ID NOs.: 19-34, respectively.

FIG. 10. A table showing AR and ERG ChIP-Seq libraries with their corresponding sequencing depth and peak numbers under different FDR calling.

FIG. 11. A table showing HDAC1, HDAC2, HDAC3, and EZH2 ChIP-Seq libraries with their corresponding sequencing depth and peak numbers under different FDR calling.

FIG. 12. A table listing Oncomine concepts that are related with ERG-associated androgen-induced gene set.

FIG. 13. Keratin genes are regulated by AR and ERG. (A) Snapshot of AR and ERG binding events near KRT8 and KRT18. (B) VCaP cells were transfected twice with control siRNA or siRNA targeting AR/ERG. Cells were then grown in 10% CDFBS before stimulated for 8 hrs with ETOH or 10 nM DHT. Afterwards, cells were harvested for total RNA, converted to cDNA before quantifying for gene expression levels. GAPDH was used as an internal normalization control. Error bars represent S.E.M of at least 3 independent experiments

FIG. 14. Vinculin expression in different clinical prostate cancer studies. (Upper panels) Boxplots showing the relative mRNA expression of Vinculin (VCL) in clinical prostate samples from the MSKCC study and several studies deposited in the Oncomine database. (Lower panels) Scatterplots showing the relative mRNA expression of VCL and its corresponding ERG mRNA expression in clinical prostate samples from the above studies.

### DETAILED DESCRIPTION

The methods and uses described herein relate to the vinculin (also referred to herein as VCL) protein, and identification of its role as a potential biological prostate cancer marker and tumour suppressor in prostate cancer cells.

VCL is a membrane cytoskeletal protein that is required for regulating focal adhesion turnover, a process that is important for proper cell movement (35). Moreover, VCL was recently shown to interact with the MET mediator, E-Cadherin, to enhance mechanosensing (36). Without being bound by theory, disruption of cell adhesion regulation may influence tumourigenesis, as well as the level of invasiveness of a tumour.

The present methods and uses relate to the discovery that vinculin expression is regulated by a particular group of transcriptional corepressors that act in the androgen regulatory pathway. That is, in prostate cancer cells, transcription factors ERG, EZH2 and various of the HDAC proteins act together to down-regulate expression of VCL. It appears that ERG may promote prostate cancer progression by working together with HDACs and EZH2 to directly modulate the transcriptional output of AR, including expression levels of VCL.

By increasing expression levels of vinculin in a prostate cancer cell, the invasiveness of the prostate cancer cell may be modulated. Similarly, levels of vinculin expressed in a given prostate cancer cell may be used as a biomarker to assess the disease state of the cancer cell (including in terms of invasiveness) and to predict prostate cancer progression.

Thus, techniques to increase vinculin expression in prostate cancer cells, including prostate cancer cells in which the vinculin expression levels are identified as lower than in healthy prostate cancer cells, may be used treat prostate cancer, including invasive prostate cancer. In some instances, techniques that target the transcriptional corepressors ERG, EZH2, HDAC1, HDAC2 and HDAC3 (including any combination thereof) may result in increased expression of vinculin in the prostate cancer cell.

Briefly, the androgen receptor (AR) is a nuclear hormone receptor. AR appears to play a central role in the progression of many prostate cancers, and androgen ablation therapy is a common treatment for many prostate cancers. AR, together with various coregulatory proteins, directs transcriptional regulation of genes involved in cell cycle, cell proliferation, survival, and differentiation. Aberrant regulation and/or expression of AR coregulatory proteins, which include specific coactivators, corepressors, collaborative factors, etc., can result in deregulation of the AR transcriptional network, resulting in uncontrolled proliferation of prostate cells.

The majority of prostate cancers harbor recurrent fusion transcripts between the promoter region of the AR direct target gene, TMPRSS2, with different ETS family members, resulting in the androgen stimulation and over-expression of ETS transcription factors (5, 6). The most important and common ETS fusion transcription factor appears to be TMPRSS2:ERG, which is detected in approximately half of all analyzed localized prostate cancers (6). ERG was shown to attenuate AR-dependent transcription (9, 10), suggesting that the induction of ERG by AR could possibly feedback to deregulate the AR transcriptional network.

Transcriptional corepressors that are associated with malignancy such as histone deacetylases (HDACs) and histone methyltransferases (HMTs) are also commonly over-expressed in prostate cancers (11, 12). HDACs are a class of enzymes that regulate the transcription of target genes by catalyzing the removal of acetyl groups from either transcription factors or the tails of histones (13). HDAC1, HDAC2 and HDAC3 are frequently over-expressed and have been shown to promote metastasis in prostate cancers (11, 14). Interestingly, high levels of HDAC1 coupled with a low expression of its target genes are key characteristics of TMPRSS2-ERG fusion positive prostate cancers (15, 16). Furthermore, ERG fusion positive prostate cancer cells are exceptionally sensitive to HDAC inhibitors (17). Consequently, epigenetic reprogramming through HDAC1 was proposed as a possible mechanism by which ERG fusion positive prostate cancers drive oncogenicity (17). The HMT, enhancer of zeste homolog 2 (EZH2), is a member of the polycomb group family that suppresses gene transcription by catalyzing the trimethylation of histone H3K27 at the promoter of target genes (18, 19). High levels of EZH2 are commonly found in invasive and hormone refractory prostate cancers (12). Recent studies suggest EZH2 can promote prostate cancer progression and contribute to metastasis by repressing the expression of developmental regulators and tumor suppressors, as well as activating the cellular de-differentiation program which results in the maintenance of prostate cancer cells in stem-cell-like state (10, 20-22).

In developing the present invention, an examination of clinical data in the Oncomine database revealed that the mRNA expression of VCL tends to be low in primary prostate cancers and may be even lower in advanced metastatic counterparts (see Examples set out below). In addition, there tends to be a negative correlation relationship between the mRNA levels of ERG and VCL, supporting the observations that ERG inhibits the androgen up-regulation of VCL expression. Finally, based on survival analysis using data from the Taylor et al clinical study (37), patients with low expression of VCL may tend to have a significantly lower recurrence free survival.

Thus, there is presently provided a method of modulating a prostate cancer cell, including modulating invasive growth of the prostate cancer cell, which involves augmenting levels of vinculin in the prostate cancer cell.

A “prostate cancer cell” refers to a prostate cell that has lost regulatory control of cell growth and proliferation, apoptosis and movement regulation, and has become transformed, including becoming malignant. The prostate cancer cell may be a cell that, when in a subject, has a tendency to migrate and invade surrounding tissue, and thus may be predisposed or have the ability to become invasive, even if currently is non-invasive. The prostate cancer cell may be a cell that, when in a subject, may be metastatic, may be predisposed or have the ability to become metastatic, even if currently non-metastatic.

The prostate cancer cell may be any prostate cancer cell, including a cell removed from a subject known or suspected to have prostate cancer, a cell contained within a subject known or suspected to have prostate cancer, for which the subject may be a human or non-human animal. The prostate cancer cell may be a cell in culture, including a prostate cancer cell from an established prostate cancer cell line or from primary tissue culture.

The term “cell” as used herein refers to and includes a single cell, a plurality of cells or a population of cells where context permits, unless otherwise specified. The cell may be an in vitro cell including a cell explanted from a subject or it may be an in vivo cell. Similarly, reference to “cells” also includes reference to a single cell where context permits, unless otherwise specified.

In some embodiments, the prostate cancer cell has reduced levels of vinculin as compared to a healthy, non-cancerous prostate cell. In some embodiments, the prostate cancer cell has been identified as having such reduced levels of vinculin.

For example, a prostate cancer cell may be tested for vinculin levels by testing a sample containing the prostate cancer cell or a prostate cancer cell derived from the same source or population of prostate cancer cells, using standard laboratory methods. Such standard methods include, for example, methods of detecting and measuring protein levels, such as chromatography methods, antibody-based methods such as immunoprecipitation, immunoblotting, ELISA, etc., including first labeling proteins within the cell with a detectable marker. Such standard methods also include, for example, methods of detecting and measuring mRNA levels or cDNA, such as PCR, sequencing, hybridization techniques, microarray techniques, methods using nucleotide probes, including first labeling the probes with a detectable marker.

The levels of vinculin in the cell, once measure, can then be compared with vinculin levels measured in a prostate cell that is non-cancerous to determine if the levels are reduced in the prostate cancer cell.

The method involves modulating invasiveness of the prostate cancer cell. Modulating or modulation of the prostate cancer cell includes reducing, slowing, limiting or halting the rate or extent of invasiveness of the prostate cancer cell into surrounding tissue, or the rate or extent of metastasis of the prostate cancer cell. Modulating or modulation may also include restoration of normal regulatory mechanisms, including regulation of movement control.

The modulating of the prostate cancer cell is achieved by augmenting the levels of expressed vinculin protein in the cell. That is, the prostate cancer cell may already express some level of endogenous vinculin. Thus, “augment”, “augmenting” or “augmentation” as used herein refers to increasing the levels of vinculin within the prostate cancer cell above the levels normally seen in the cell in the absence of augmentation.

Augmenting the levels of vinculin may be achieved by administering to the prostate cancer cell an agent that augments levels of vinculin expression.

For example, the agent may result in augmenting the levels of vinculin in the prostate cancer cell expressed from the endogenous vinculin gene by chemical or by genetic methods, including by exposure to a chemical or compound that increases expression of vinculin or by introducing a nucleic acid molecule or expression cassette encoding a regulatory factor that results in increased expression of native vinculin in the prostate cancer cell.

Chemicals or compounds that increase the level of vinculin expression in a cell include chemicals or compounds that inhibit the activity or expression of one or more of the following proteins: ERG, EZH2, HDAC1, HDAC2 and HDAC3. Thus, a single agent may be administered to the prostate cell, to result in inhibition of any one of ERG, EZH2, HDAC1, HDAC2 and HDAC3. As well, a single agent or a combination of agents may be administered to the prostate cell, to result in a combined inhibition of any combination of ERG, EZH2, HDAC1, HDAC2 and HDAC3.

The agent may be a chemical agent that inhibits the activity of one or more of ERG, EZH2, HDAC1, HDAC2 and HDAC3. Chemicals that inhibit one or more of ERG, EZH2, HDAC1, HDAC2 and HDAC3 include 3-deazaneplanocin A (DZNep) and Trichostatin A (TSA). Inhibitors may include sinefungin, adenosine dialdehyde and 5-aza-2′-deoxycytidine.

In order to effect the augmentation, the prostate cancer cell thus may be exposed to the chemical agent or combination of agents that inhibits one or more of ERG, EZH2, HDAC1, HDAC2 and HDAC3. For example, the chemical agent or combination of chemical agents may be contacted with the prostate cancer cell such that the chemical agent is taken up by or is able to enter into the prostate cancer cell.

Alternatively, the agent may be an agent that inhibits the expression of one or more of ERG, EZH2, HDAC1, HDAC2 and HDAC3.

For example, the agent may be an antisense RNA molecule or an siRNA or a combination of antisense RNA molecules or siRNAs directed against the mRNA of one or more of ERG, EZH2, HDAC1, HDAC2 and HDAC3.

The antisense RNA molecule will contain a sequence that is complementary to at least a fragment of an RNA transcript of a gene encoding the relevant protein of ERG, EZH2, HDAC1, HDAC2 and HDAC3, and which can bind to the transcript of such gene, thereby reducing or preventing the expression of the gene encoding ERG, EZH2, HDAC1, HDAC2 or HDAC3 in vivo. The antisense RNA molecule should have a sufficient degree of complementarity to the target mRNA to avoid non-specific binding of the antisense molecule to non-target sequences under conditions in which specific binding is desired, such as under physiological conditions.

The siRNA molecule may be any double-stranded RNA molecule, including a self-complementary single-stranded molecule that can fold back on itself to form the double-stranded siRNA, which induces gene-specific RNA interference in a cell, leading to decreased or no expression of a gene encoding ERG, EZH2, HDAC1, HDAC2 or HDAC3 in vivo. An siRNA typically targets a 19-23 base nucleotide sequence in a target mRNA, as described in Elbashir, et al. (2001) EMBO J 20: 6877-6888, the contents of which is incorporated herein by reference.

In order to effect the augmentation, the prostate cancer cell thus may be exposed to the antisense RNA, a nucleotide encoding the antisense RNA, the siRNA or a nucleotide encoding the siRNA, for example a nucleic acid vector containing a nucleic acid molecule which allows for transcription of an antisense transcript or a single-stranded, self-complementary siRNA molecule capable of forming a double-stranded siRNA. Such an antisense molecule, siRNA molecule or vector may be synthesized using nucleic acid chemical synthesis methods and standard molecular biology cloning techniques.

In another example, the agent may be a DNA enzyme or a combination of DNA enzymes directed against the mRNA of one or more of ERG, EZH2, HDAC1, HDAC2 and HDAC3. A DNA enzyme is a magnesium-dependent catalytic nucleic acid composed of DNA that can selectively bind to an RNA substrate by Watson-Crick base-pairing and potentially cleave a phosphodiester bond of the backbone of the RNA substrate at any purine-pyrimidine junction (Santiago, F. S., et al., (1999) Nat Med 5: 1264-1269). A DNA enzyme is composed of two distinct functional domains: a 15-nucleotide catalytic core that carries out phosphodiester bond cleavage, and two hybridization arms flanking the catalytic core; the sequence identity of the arms can be tailored to achieve complementary base-pairing with target RNA substrates.

The DNA enzyme will therefore have complementary regions that can anneal with regions on the transcript of a gene encoding ERG, EZH2, HDAC1, HDAC2 or HDAC3, flanking a purine-pyrimidine junction such that the catalytic core of the DNA enzyme is able to cleave the transcript at the junction, rendering the transcript unable to be translated to produce a functional protein. In certain embodiments, the DNA enzyme is designed to cleave the mRNA transcript of the relevant ERG, EZH2, HDAC1, HDAC2 or HDAC3 gene between the A and the U residues of the AUG start codon.

The DNA enzyme may be synthesized using standard techniques known in the art, for example, standard phosphoramidite chemical ligation methods may be used to synthesize the DNA molecule in the 3′ to 5′ direction on a solid support, including using an automated nucleic acid synthesizer. Alternatively, the DNA enzyme may be synthesized by transcribing a nucleic acid molecule encoding the DNA enzyme. The nucleic acid molecule may be contained within a DNA or RNA vector, for delivery into a cellular expression system, for example, a viral vector. Suitable viral vectors include vaccinia viral vectors and adenoviral vectors.

Thus, the augmentation may be achieved by exposing the prostate cancer cell to the DNA enzyme so that the DNA enzyme is taken up by the cell, and is able to target and cleave the relevant ERG, EZH2, HDAC1, HDAC2 or HDAC3 transcript in the cell, resulting in decreased or no expression of functional protein in the cell. Exposure may include transfection techniques, as are known in the art, or by microinjection techniques in which the DNA is directly injected into the cell. Exposure may also include exposing the cell to the naked DNA enzyme, as cells may take up naked DNA in vivo. Alternatively, if the DNA enzyme is included in a nucleic acid vector, such as a viral vector, the cell may be infected with the viral vector.

Augmenting the levels of vinculin in a prostate cancer cell also includes genetically modifying the prostate cancer cell to include an exogenous nucleic acid molecule encoding vinculin, including an expression cassette comprising the vinculin coding region. It will be understood that for vinculin to be expressed in the genetically modified prostate cancer cell, the nucleic acid molecule will contain the coding region of vinculin operably linked to the necessary regulatory regions required to effect expression, including a suitable native or heterologous promoter region and optionally enhancer elements. As stated above, the prostate cancer cell may already express vinculin, and therefore the nucleic acid molecule encoding vinculin may be designed to result in total expression levels of vinculin in the prostate cancer cell at levels above the natural levels of expression in the prostate cancer cell in the absence of augmentation.

Genetic modification can be achieved using molecular biology and cloning methods known in the art. For example, the prostate cancer cell may be transformed or transfected with a vector designed to express vinculin from a native or heterologous promoter, and the promoter may be a constitutive, transient or inducible promoter, and may direct expression at basal or heightened levels of expression. Suitable vectors include bacterial plasmids or viral vectors including viral genomes. For example, a baculoviral, a retroviral, a lentiviral or an Adenoviral vector may be used.

The vinculin protein that is expressed using genetic modification techniques may be any vinculin protein that, when expressed in a prostate cancer cell at augmented levels, that is to raise the overall level of vinculin in the prostate cancer cell above levels seen with native expression of vinculin within that cell, modulates the invasiveness of the prostate cancer cell, as indicated above.

For example, the vinculin protein may comprise, consist or consist essentially of the sequence of human vinculin isoform 2 [SEQ ID NO.: 1]:

MPVFHTRTIESILEPVAQQISHLVIMHEEGEVDGKAIPDLTAPVAAVQA
AVSNLVRVGKETVQTTEDQILKRDMPPAFIKVENACTKLVQAAQMLQSD
PYSVPARDYLIDGSRGILSGTSDLLLTFDEAEVRKIIRVCKGILEYLTV
AEVVETMEDLVTYTKNLGPGMTKMAKMIDERQQELTHQEHRVMLVNSMN
TVKELLPVLISAMKIFVTTKNSKNQGIEEALKNRNFTVEKMSAEINEII
RVLQLTSWDEDAWASKDTEAMKRALASIDSKLNQAKGWLRDPSASPGDA
GEQAIRQILDEAGKVGELCAGKERREILGTCKMLGQMTDQVADLRARGQ
GSSPVAMQKAQQVSQGLDVLTAKVENAARKLEAMTNSKQSIAKKIDAAQ
NWLADPNGGPEGEEQIRGALAEARKIAELCDDPKERDDILRSLGEISAL
TSKLADLRRQGKGDSPEARALAKQVATALQNLQTKTNRAVANSRPAKAA
VHLEGKIEQAQRWIDNPTVDDRGVGQAAIRGLVAEGHRLANVMMGPYRQ
DLLAKCDRVDQLTAQLADLAARGEGESPQARALASQLQDSLKDLKARMQ
EAMTQEVSDVFSDTTTPIKLLAVAATAPPDAPNREEVFDERAANFENHS
GKLGATAEKAAAVGTANKSTVEGIQASVKTARELTPQVVSAARILLRNP
GNQAAYEHFETMKNQWIDNVEKMTGLVDEAIDTKSLLDASEEAIKKDLD
KCKVAMANIQPQMLVAGATSIARRANRILLVAKREVENSEDPKFREAVK
AASDELSKTISPMVMDAKAVAGNISDPGLQKSFLDSGYRILGAVAKVRE
AFQPQEPDFPPPPPDLEQLRLTDELAPPKPPLPEGEVPPPRPPPPEEKD
EEFPEQKAGEVINQPMMMAARQLHDEARKWSSKPGIPAAEVGIGVVAEA
DAADAAGFPVPPDMEDDYEPELLLMPSNQPVNQPILAAAQSLHREATKW
SSKGNDIIAAAKRMALLMAEMSRLVRGGSGTKRALIQCAKDIAKASDEV
TRLAKEVAKQCTDKRIRTNLLQVCERIPTISTQLKILSTVKATMLGRTN
ISDEESEQATEMLVHNAQNLMQSVKETVREAEAASIKIRTDAGFTLRWV
RKTPWYQ

For example, the vinculin protein may comprise, consist or consist essentially of the sequence of human vinculin isoform 1 [SEQ ID NO.: 2]:

MPVFHTRTIESILEPVAQQISHLVIMHEEGEVDGKAIPDLTAPVAAVQA
AVSNLVRVGKETVQTTEDQILKRDMPPAFIKVENACTKLVQAAQMLQSD
PYSVPARDYLIDGSRGILSGTSDLLLTFDEAEVRKIIRVCKGILEYLTV
AEVVETMEDLVTYTKNLGPGMTKMAKMIDERQQELTHQEHRVMLVNSMN
TVKELLPVLISAMKIFVTTKNSKNQGIEEALKNRNFTVEKMSAEINEII
RVLQLTSWDEDAWASKDTEAMKRALASIDSKLNQAKGWLRDPSASPGDA
GEQAIRQILDEAGKVGELCAGKERREILGTCKMLGQMTDQVADLRARGQ
GSSPVAMQKAQQVSQGLDVLTAKVENAARKLEAMTNSKQSIAKKIDAAQ
NWLADPNGGPEGEEQIRGALAEARKIAELCDDPKERDDILRSLGEISAL
TSKLADLRRQGKGDSPEARALAKQVATALQNLQTKTNRAVANSRPAKAA
VHLEGKIEQAQRWIDNPTVDDRGVGQAAIRGLVAEGHRLANVMMGPYRQ
DLLAKCDRVDQLTAQLADLAARGEGESPQARALASQLQDSLKDLKARMQ
EAMTQEVSDVFSDTTTPIKLLAVAATAPPDAPNREEVFDERAANFENHS
GKLGATAEKAAAVGTANKSTVEGIQASVKTARELTPQVVSAARILLRNP
GNQAAYEHFETMKNQWIDNVEKMTGLVDEAIDTKSLLDASEEAIKKDLD
KCKVAMANIQPQMLVAGATSIARRANRILLVAKREVENSEDPKFREAVK
AASDELSKTISPMVMDAKAVAGNISDPGLQKSFLDSGYRILGAVAKVRE
AFQPQEPDFPPPPPDLEQLRLTDELAPPKPPLPEGEVPPPRPPPPEEKD
EEFPEQKAGEVINQPMMMAARQLHDEARKWSSKGNDIIAAAKRMALLMA
EMSRLVRGGSGTKRALIQCAKDIAKASDEVTRLAKEVAKQCTDKRIRTN
LLQVCERIPTISTQLKILSTVKATMLGRTNISDEESEQATEMLVHNAQN
LMQSVKETVREAEAASIKIRTDAGFTLRWVRKTPWYQ

For example, the vinculin protein may comprise, consist or consist essentially of the sequence of human vinculin isoform 3 [SEQ ID NO.: 3]:

MPPAFIKVENACTKLVQAAQMLQSDPYSVPARDYLIDGSRGILSGTSDL
LLTFDEAEVRKIIRVCKGILEYLTVAEVVETMEDLVTYTKNLGPGMTKM
AKMIDERQQELTHQEHRVMLVNSMNTVKELLPVLISAMKIFVTTKNSKN
QGIEEALKNRNFTVEKMSAEINEIIRVLQLTSWDEDAWASKVRVLSGEI
SKIPNSPWLGVLIGTCLILYLVIFVA

For example, the vinculin protein may comprise, consist or consist essentially of the sequence of mouse vinculin [SEQ ID NO.: 4]:

MPVFHTRTIESILEPVAQQISHLVIMHEEGEVDGKAIPDLTAPVAAVQA
AVSNLVRVGKETVQTTEDQILKRDMPPAFIKVENACTKLVQAAQMLQSD
PYSVPARDYLIDGSRGILSGTSDLLLTFDEAEVRKIIRVCKGILEYLTV
AEVVETMEDLVTYTKNLGPGMTKMAKMIDERQQELTHQEHRVMLVNSMN
TVKELLPVLISAMKIFVTTKNSKNQGIEEALKNRNFTVEKMSAEINEII
RVLQLTSWDEDAWASKDTEAMKRALASIDSKLNQAKGWLRDPNASPGDA
GEQAIRQILDEAGKVGELCAGKERREILGTCKMLGQMTDQVADLRARGQ
GASPVAMQKAQQVSQGLDVLTAKVENAARKLEAMTNSKQSIAKKIDAAQ
NWLADPNGGPEGEEQIRGALAEARKIAELCDDPKERDDILRSLGEIAAL
TSKLGDLRRQGKGDSPEARALAKQVATALQNLQTKTNRAVANSRPAKAA
VHLEGKIEQAQRWIDNPTVDDRGVGQAAIRGLVAEGHRLANVMMGPYRQ
DLLAKCDRVDQLTAQLADLAARGEGESPQARALASQLQDSLKDLKAQMQ
EAMTQEVSDVFSDTTTPIKLLAVAATAPPDAPNREEVFDERAANFENHS
GRLGATAEKAAAVGTANKSTVEGIQASVKTARELTPQVISAARILLRNP
GNQAAYEHFETMKNQWIDNVEKMTGLVDEAIDTKSLLDASEEAIKKDLD
KCKVAMANIQPQMLVAGATSIARRANRILLVAKREVENSEDPKFREAVK
AASDELSKTISPMVMDAKAVAGNISDPGLQKSFLDSGYRILGAVAKVRE
AFQPQEPDFPPPPPDLEQLRLTDELAPPKPPLPEGEVPPPRPPPPEEKD
EEFPEQKAGEVINQPMMMAARQLHDEARKWSSKGNDIIAAAKRMALLMA
EMSRLVRGGSGTKRALIQCAKDIAKASDEVTRLAKEVAKQCTDKRIRTN
LLQVCERIPTISTQLKILSTVKATMLGRTNISDEESEQATEMLVHNAQN
LMQSVKETVREAEAASIKIRTDAGFTLRWVRKTPWYQ

The term “consists essentially of” or “consisting essentially of” as used herein means that a molecule may have additional features or elements beyond those described provided that such additional features or elements do not materially affect the ability of the molecule to function as an analyte molecule or a capture molecule, as the case may be. That is, the molecule may have additional features or elements that do not interfere with the activity of the molecule. For example, a peptide or protein consisting essentially of a specified sequence may contain one, two, three, four, five or more additional amino acids, at one or both ends of the sequence provided that the additional amino acids do not inhibit, block, interrupt or interfere with the activity of the peptide or protein. In a further example, a nucleic acid molecule consisting essentially of a specified nucleotide sequence may contain one, two, three, four, five or more nucleotides at one or both ends of the specified sequence provided the nucleic acid molecule can still function as intended. Similarly, a peptide, protein or nucleic acid molecule may be chemically modified with one or more functional groups provided that such chemical groups do not interfere with the activity of the molecule.

Thus, the vinculin protein includes homologues of vinculin, and any derivative, variant, or fragment thereof that when expressed in a prostate cancer cell at augmented levels, modulates the invasiveness of the prostate cancer cell, as indicated above. A polynucleotide sequence or polypeptide sequence is a “homologue” of, or is “homologous” to, another sequence if the two sequences have substantial identity over a specified region and the functional activity of the sequences is conserved (as used herein, the term ‘homologous’ does not infer evolutionary relatedness). Two polynucleotide sequences or polypeptide sequences are considered to have substantial identity if, when optimally aligned (with gaps permitted), they share at least about 50% sequence identity, or if the sequences share defined functional motifs. In alternative embodiments, optimally aligned sequences may be considered to be substantially identical (i.e. to have substantial identity) if they share at least 60%, 70%, 75%, 80%, 85%, 90%, 95%, 96%, 97%, 98%, 99 identity over a specified region. An “unrelated” or “non-homologous” sequence shares less than 40% identity, though preferably less than about 25% identity, with a polypeptide or polynucleotide of the invention over a specified region of homology. The terms “identity” and “identical” refer to sequence similarity between two peptides or two polynucleotide molecules. Identity can be determined by comparing each position in the aligned sequences. A degree of identity between amino acid sequences is a function of the number of identical or matching amino acids at positions shared by the sequences, i.e. over a specified region. Optimal alignment of sequences for comparisons of identity may be conducted using a variety of algorithms, as are known in the art, including the ClustalW program, available at clustalw.genome.ad.jp, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at www.ncbi.nlm.nih.gov).

A variant or derivative of vinculin refers to a fragment, either alone or contained in a fusion or chimeric protein, which retains the ability to modulate the invasiveness of a prostate cancer cell when expressed in the prostate cancer cell at augmented levels, or a vinculin protein that has been mutated at one or more amino acids, including point, insertion or deletion mutation, but still retains the ability to direct migration of a neural precursor cell toward a glioma cell or a cell that secretes a neural precursor cell chemoattractant factor, as well as non-peptides and peptide mimetics which possess the ability to mimic the biological activity of vinculin. A variant or derivative therefore includes deletions, including truncations and fragments; insertions and additions, including tagged polypeptides and fusion proteins; substitutions, for example conservative substitutions, site-directed mutants and allelic variants; and modifications, including peptoids having one or more non-amino acyl groups (q.v., sugar, lipid, etc.) covalently linked to the peptide and post-translational modifications. As used herein, the term “conserved amino acid substitutions” or “conservative substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. Conservative changes can also include the substitution of a chemically derivatised moiety for a non-derivatised residue, for example, by reaction of a functional side group of an amino acid.

Variants and derivatives can be prepared, for example, by substituting, deleting or adding one or more amino acid residues in the amino acid sequence of a vinculin protein or fragment thereof, and screening for biological activity. Preferably, substitutions are made with conservative amino acid residues, i.e., residues having similar physical, biological or chemical properties. A skilled person will understand how to make such derivatives or variants, using standard molecular biology techniques and methods, described for example in Sambrook et al. ((2001) Molecular Cloning: a Laboratory Manual, 3^{rd} ed., Cold Spring Harbour Laboratory Press), and how to test such derivatives or variants for their ability to modulate the invasiveness of a prostate cancer cell when expressed in the prostate cancer cell.

As indicated above, in some embodiments the cell may be an in vivo cell in a subject. In such a case, if the subject is to be treated, the agent may be formulated in a pharmaceutical composition for administration to the subject.

The subject may be any subject who is in need of treatment of prostate cancer, for example a subject who has been diagnosed with prostate cancer or who is predisposed to developing prostate cancer or who is suspected of having prostate cancer. As indicated above, the prostate cancer may be at any stage of development, may be invasive or non-invasive, may be metastatic or non-metastatic.

The term “treating” prostate cancer refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total). “Treating” can also mean prolonging survival of a patient beyond that expected in the absence of treatment. “Treating” can also mean inhibiting the progression of disease, slowing the progression of disease temporarily, although more preferably, it involves halting the progression of the disease permanently.

Thus, when the prostate cancer cell is in a subject, administering the agent to the prostate cancer cell to augment levels of vinculin may be used to treat the subject.

An effective amount of the agent is administered to the subject. The term “effective amount” as used herein means an amount effective, at dosages and for periods of time necessary to achieve the desired result, for example, to treat the prostate cancer.

The agent may be administered to the patient using standard techniques known in the art. The molecule may be administered systemically, or may be administered directly at the site of the prostate cancer. Delivery to the site includes topical administration, injection to the site, or surgical implantation, for example at a site of a tumour.

The concentration and amount of the agent that augments the vinculin levels in a prostate cancer cell that is to be administered will vary, depending on the nature of the prostate cancer that is to be treated, the type of molecule that is administered, the mode of administration, and the age and health of the subject.

The dose of the pharmaceutical composition that is to be used depends on a variety of factors, including the prostate cancer being treated, the severity of the condition, the individual patient parameters including age, physical condition, size and weight, the duration of the treatment, the nature of concurrent therapy (if any), the specific route of administration and other similar factors that are within the knowledge and expertise of the health practitioner. These factors are known to those of skill in the art and can be addressed with minimal routine experimentation.

As indicated above, to aid in administration, the agent that augments vinculin levels may be formulated as an ingredient in a pharmaceutical composition.

Therefore, there is also provided a pharmaceutical composition comprising an agent that augments vinculin expression levels in a prostate cancer cell. The invention in one aspect therefore also includes such pharmaceutical compositions for use in treating prostate cancer. The compositions may routinely contain pharmaceutically acceptable concentrations of salt, buffering agents, preservatives and various compatible carriers. For all forms of delivery, the agent that augments vinculin levels in a prostate cell may be formulated in a physiological salt solution.

The pharmaceutical compositions may additionally contain other therapeutic agents useful for treating prostate cancer, for example a cytotoxic agent, for example a chemotherapeutic agent.

The pharmaceutical composition may optionally contain a pharmaceutically acceptable diluent. The proportion and identity of the pharmaceutically acceptable diluent may be determined by chosen route of administration, and standard pharmaceutical practice. Generally, the pharmaceutical composition will be formulated with components that will not significantly impair the biological, chemical or pharmaceutical properties of the agent. Suitable diluents are described, for example, in Remington's Pharmaceutical Sciences (Remington's Pharmaceutical Sciences, Mack Publishing Company, Easton, Pa., USA 1985). On this basis, the pharmaceutical compositions include, albeit not exclusively, solutions of the agents that augment vinculin levels in a prostate cell, in association with one or more pharmaceutically acceptable vehicles or diluents, and contained in buffer solutions with a suitable pH and iso-osmotic with physiological fluids.

Also contemplated are uses of the described agents that augment vinculin expression levels in the treatment of prostate cancer, including for use in the manufacture of a medicament for treatment of prostate cancer.

Vinculin may also be used as a biomarker to assess prostate cancer disease state and progression in a subject. As set out in the examples below, vinculin levels have often found to be low in prostate cancers, which appears to be a result of the coordinated actions of AR and ERG recruiting corepressor proteins EZH2, HDAC1, HDAC2 and HDAC3, resulting in targeting of the vinculin gene.

Thus, by detecting vinculin levels in a prostate cancer cell, prostate cancer disease status may be monitored.

Prostate cancer disease status includes current disease state, the rate of disease progression, invasiveness or likelihood of invasiveness of a tumour, as well as prognosis for disease, response to treatment or likelihood of response to treatment.

Thus, there is provided a method of diagnosis prostate cancer disease status in a subject. The method includes detecting the vinculin levels, including vinculin protein, mRNA or cDNA levels, in a prostate cancer cell that has been obtained from the subject.

The vinculin levels are detected using standard methods for determining protein levels in a cell. For example, methods for detecting protein levels include chromatography methods, antibody-based methods such as immunoprecipitation, immunoblotting, ELISA, etc., including first labeling proteins within the cell with a detectable marker. Methods for detecting mRNA or cDNA levels include PCR methods, sequencing methods, hybridization methods, microarray methods, methods using nucleotide probes, including first labeling the probes with a detectable marker.

The levels of vinculin in the sample from the subject are compared with vinculin levels in a prostate cell sample obtained from a healthy prostate cell that is not cancerous. Levels of vinculin lower in the sample from the subject than that in a healthy prostate cell may be indicative that the sample from the subject is cancerous and may be indicative that the sample is an invasive cancer or has a predisposition to become an invasive cancer. Levels of vinculin lower in the sample from the subject than that in a healthy prostate cell may be indicative that the subject may be treated by augmenting levels of vinculin in the subject's prostate cancer cells.

The prostate cancer cell may be first obtained from the patient, using standard methods, including biopsy techniques for removing a cancer sample from a subject.

The present methods and uses are further exemplified by way of the following non-limiting examples.

### EXAMPLES

### Example 1

Transcriptional corepressors are frequently aberrantly over-expressed in prostate cancers. However, crosstalk between the corepressors and the Androgen receptor (AR), the key player in prostate cancer development, is largely unclear. In this experiment, using chromatin immunoprecipitation coupled to massively parallel sequencing (ChIP-Seq), global binding maps of AR, ERG, and commonly over-expressed transcriptional corepressors in prostate cancer cells were generated, including HDAC1, HDAC2, HDAC3, and EZH2 before and after androgen stimulation. The results demonstrate that ERG, HDACs, and EZH2 are directly involved in androgen-regulated transcription and wired into an AR centric transcriptional network via a spectrum of distal enhancers and/or proximal promoters. Moreover, the results showed these corepressors function as a multi-protein complex to enhance ERG-mediated repression of AR-induced transcription including cytoskeletal genes that promote epithelial differentiation and inhibit metastasis. Specifically, it was demonstrated that the direct suppression of Vinculin expression by ERG, EZH2, and HDACs lead to increased cancer cell invasiveness. Taken together, these results highlight a novel mechanism by which the fusion gene product, ERG, working together with oncogenic corepressors including HDACs and the polycomb protein, EZH2, could impede epithelial differentiation and contribute to prostate cancer progression, through directly modulating the transcriptional output of AR.

Materials and Methods

Cell Culture:

The human prostate cancer cell line, VCaP (American Type Culture Collection), was maintained in DMEM supplemented with 10% fetal bovine serum (FBS), sodium pyruvate, sodium bicarbonate, and penicillin/streptomycin at 37° C. under 5% CO2. Unless otherwise stated, for experiments requiring DHT (Tokyo Chemical Industry) treatment, VCaP cells were grown for 24 hours prior to stimulation in phenol red free DMEM supplemented with 10% charcoal-dextran stripped fetal bovine serum (CDFBS), sodium pyruvate, sodium bicarbonate, and penicillin/streptomycin.

Chromatin Immunoprecipitation (ChIP):

ChIP assay was performed as described previously (47). Briefly, VCaP cells were fixed with 1% formaldehyde (Sigma-Aldrich) for 10 minutes prior to harvesting. Next, the cells were resuspended in SDS lysis buffer containing protease inhibitors. The lysed cells were then sonicated shearing the chromatin into 500-1000 base pairs in length. After pre-clearing with rabbit IgG (Santa Cruz Biotechnology) for 2 hours, the chromatin was immunoprecipitation overnight with the corresponding antibodies and Sepharose beads A (Zymed). After washing the beads, the captured DNA was eluted and de-crosslinked at 65° C. overnight prior to purification with QIAquick spin PCR purification kit (Qiagen). Real-time quantitative Polymerase Chain Reaction (qPCR) was carried out using the KAPA SYBR FAST qPCR kit (Kapa Biosystems).

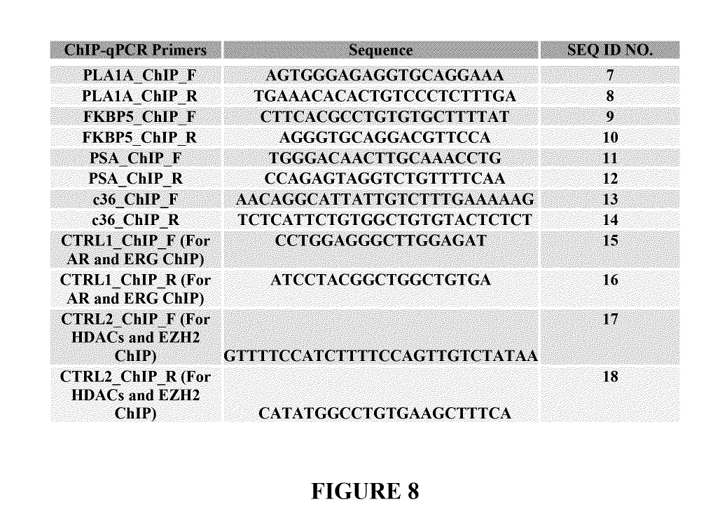

All ChIP-qPCR primer sequences can be found in FIG. 8. For HDAC1, HDAC2, HDAC3, and EZH2 ChIP, a double cross-linking strategy was used to stabilize protein-protein interactions. Specifically, cells were first fixed with 2 mM DSG (Pierce) for 45 minutes prior to formaldehyde fixation. Antibodies that were used for ChIP analysis include anti-AR (sc-815x), anti-ERG (sc-353), anti-HDAC3 (sc-11417) from Santa Cruz Biotechnology, anti-HDAC1 (ab7028-50), anti-HDAC2 (ab7029-50) from Abcam, and anti-EZH2 (39933) from Active Motif.

Short Interfering RNAs (siRNAs):

Unless otherwise stated, siRNA studies were carried out using a double knockdown approach. Briefly, VCaP cells were transfected twice with the selected siRNA at a concentration of 100 nM/transfection using Lipofectamine RNAi Max (Invitrogen) with a 24 hours interval between each transfection. The siRNAs used in this study were siAR (ON-TARGETplus SMARTpool L-003400-00), siERG (SiGENOME D-003886-01) from Dharmacon, and siVCL synthesized from 1stBase. The siVCL sequence is rCrUrGrGrCrUrUrGrCrArGrArUrCrCrArArArUrUrU [SEQ ID NO.: 5]. The control siRNA for the siAR and siERG experiments was from Dharmacon (D-001206-13), while the control siRNA for the siVCL experiments was from 1stBase (rUrUrCrUrCrCrGrArArCrGrUrGrUrCrArCrGrUrTrT [SEQ ID NO.: 6]).

Gene Expression Analysis:

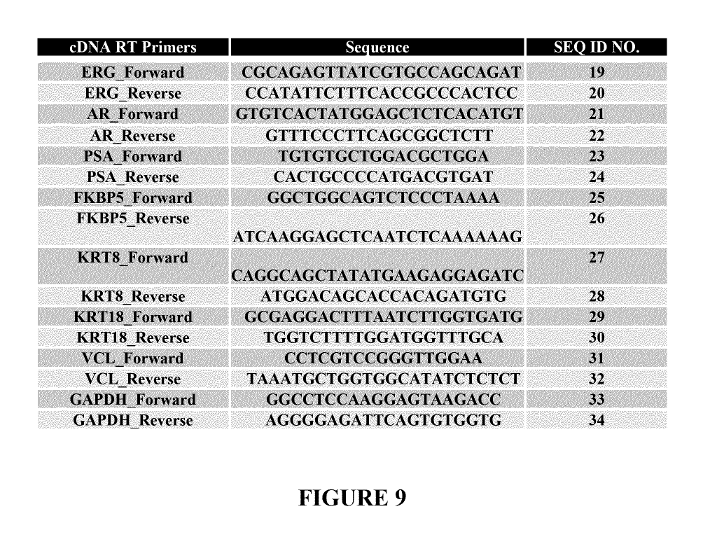

Cells were harvested and total RNA was collected in TRI-reagent (Sigma) and purified with PureLink™ RNA Mini Kit (Invitrogen). Reverse transcription of RNA to cDNA was carried out using M-MLV reverse transcriptase (Promega). RNA expression levels were measured using quantitative PCR and normalized to GAPDH. The primers for cDNA quantification can be found in FIG. 9.

Microarray Expression Profiling:

Purified total RNA from three independent biological replicates of VCaP cells exposed to varying lengths of DHT stimulation were converted to cRNA using the Illumina® TotalPrep™-96 RNA Amplification Kit (Ambion) according to the manufacturer's instructions. cRNA was hybridized onto Sentrix® HumanRef-8 v3 Expression BeadChip Kit (Illumina). The BeadChips were scanned with the BeadArray Reader and the image data was processed using GenomeStudio. The gene expression data was analyzed using GeneSpring GX 11.0 software.

Co-Immunoprecipitation:

All co-IP experiments were performed with VCaP cells grown in full serum conditions. VCaP cells were trypsinized and lysed to obtain whole cell lysate. The cell lysate was subsequently pre-cleared with Protein A/G-Agarose beads (Roche Applied Science) at 4° C. for 4 hrs. An aliquot of the whole cell lysate was collected and stored at −80° C. as input for the western blot analysis. After pre-clearing, the supernatant was incubated overnight at 4° C. with 5 μg of anti-AR (sc-815x) or anti-ERG (sc-353). Roche beads were added into the mixture on the next day and incubated for 1.5 hrs at 4° C., and then washed with TBS for four times. Finally, the beads were heated to 99° C. for 5 minutes and eluted with SDS loading buffer for western blot analysis.

Western Blot Analysis:

The antibodies used for western blot analysis include anti-AR (sc-816), anti ERG (sc-354), anti-Vinculin (sc-25336) from Santa Cruz, anti-AR (AR441) from Labvision, anti-HDAC1 (#05-100), anti-HDAC2 (#05-814) from Millipore, anti-HDAC3 (#3949) and anti-EZH2 (#3147) from Cell Signaling Technology.

ChIP-Seq:

Quant-iT™ PicoGreen® dsDNA assay kit (Invitrogen, Molecular Probes) was used to quantify 5 to 10 ng of ChIP DNA for library construction. Libraries were prepared using Illumina's ChIP-seq DNA sample preparation kit with minor modifications. Briefly, amplification of adaptor-ligated DNA was performed using Pfx DNA polymerase (Invitrogen) for 15 cycles. Amplified products of 200˜300 bp were extracted for sequencing. ChIP-seq reads were aligned to the reference human genome (UCSC, hg18) and binding peaks using input reads as control were determined with Control-based ChIP-Seq Analysis Tools (CCAT) (48).

Conservation Analysis for Binding Peaks:

Conservation scores for the alignment of 27 vertebrate genomes with Human (PhastCons28way) were downloaded from the UCSC Genome Browser database. The sequence conservation score for every position in a 2000 bp window centering on the defined ChIP-Seq peak/cluster were plotted for comparison.

Generation of Heatmap Binding Signals:

ERG and AR binding peaks that were within 500 bp of each other were clustered together for the generation of the plot. For a fair comparison of tag intensity, the AR and ERG libraries were re-sampled to 10 million reads before being plotted out as binding signals around a region of −/+2 kb centralized at the respective AR/ERG ChIP-Seq peak or defined AR/ERG clusters (−/+2 kb). The individual binding region was sorted by their binding signals at their respective categories (AR only, ERG only and AR/ERG overlap) for easy visualization.

Matrigel Invasion Assay:

Invasion assay was performed using (8.0 μm pore size) HTS FluoroBlok Cell Culture Inserts (BD). Briefly, 750 μl of media (with 20% FBS) was added into each well of a 24-well plate and inserts were placed individually into each well. Each insert was first coated with 80 μl of the pre-diluted (250 μg/ml) Matrigel Basement Matrix (BD). Next, 4×105 siRNA-treated VCaP cells in 200 μl media (with 0.5% FBS) were seeded into each well. After 48 hrs, the cells at the bottom of the inserts were fixed with 3.7% formaldehyde for 15 minutes prior to staining with 25 μg/ml propidium iodide for 30 minutes in the dark. Any cells that passed through the base membrane of the inserts were then scanned by a Cellomics Arrayscan. Ten different fields were taken for each insert. Each condition was assayed in technical triplicates for biological triplicates.

Data Deposition:

Raw ChIP-Seq and gene expression profiling data generated from this study have been deposited at the NCBI GEO repository under accession number GSE28951.

Results

DHT Stimulates Differential Expression and Binding of AR and ERG at ARBS:

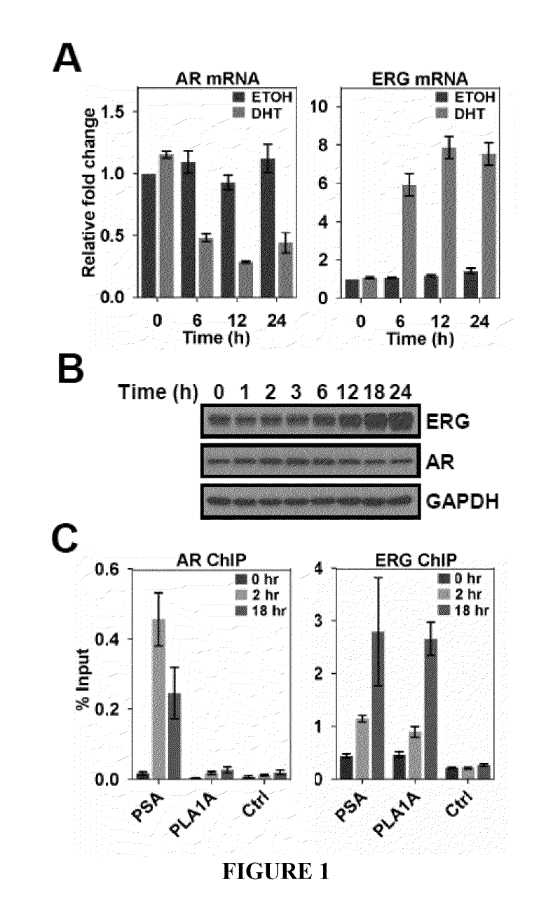

TMPRSS2:ERG is regulated by androgen stimulation and is over-expressed in the majority of prostate cancer tumors (5). Despite recent efforts to unravel the transcriptional crosstalk between AR and ERG, the underlying mechanism of how androgen signaling affects the genome-wide binding of these factors to chromatin in prostate cancer cells is unclear. To address this, we first examined the effect of DHT on the mRNA and protein expression level of AR and ERG. As shown in FIG. 1A-B, DHT repressed the level of AR mRNA across time, while the protein level remained relatively constant with only a slight decrease after long DHT exposure (FIG. 1B). In contrast, DHT up-regulated both the mRNA and protein levels of ERG although with different kinetics (peaking at 6 hrs for RNA and 12 hrs for protein) (FIG. 1A-B).

We next examined the binding of AR and ERG to chromatin upon DHT stimulation. Because of the differential expression of these proteins in response to DHT, we performed AR and ERG ChIP at various times after DHT stimulation. As shown in FIG. 1C, AR was recruited strongly to the enhancer AR binding site (ARBS) of PSA 2 hrs after DHT stimulation, however, the binding was reduced significantly after 18 hrs. In comparison, ERG was also recruited to the ARBS of PSA and to the previously identified ERG binding site (ERGBS) associated with PLA1A 2 hrs after DHT treatment, however, unlike AR, the recruitment of ERG was further enhanced after 18 hrs.

Taken together, our results demonstrate that AR and ERG can be co-localized together upon androgen signaling, but the binding kinetics of the two transcription factors to chromatin are distinct.

Global Analysis of AR and ERG Binding Sites:

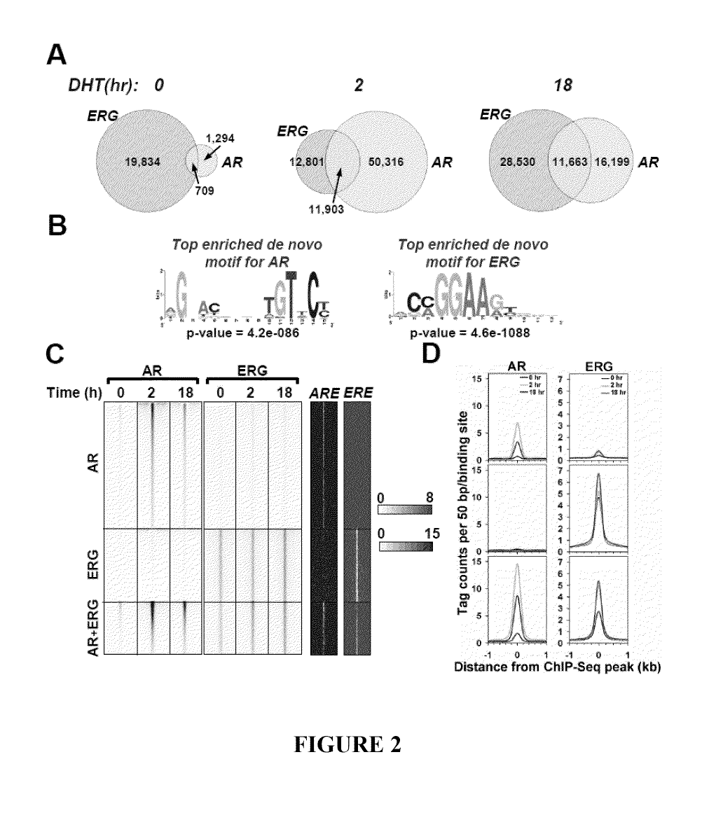

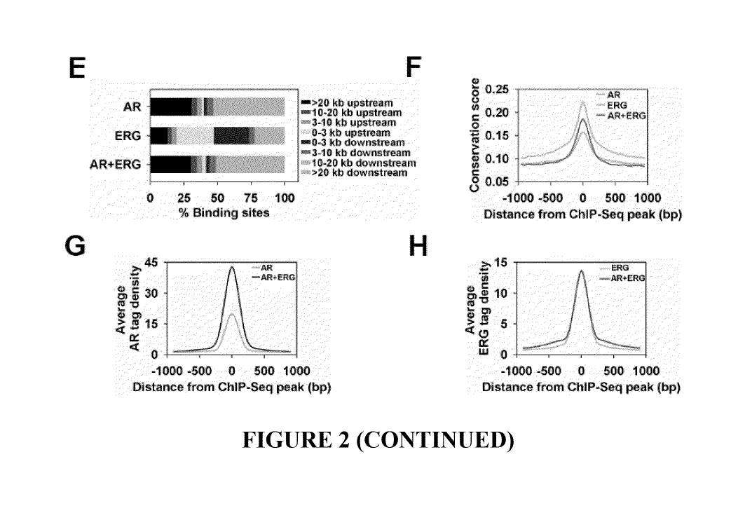

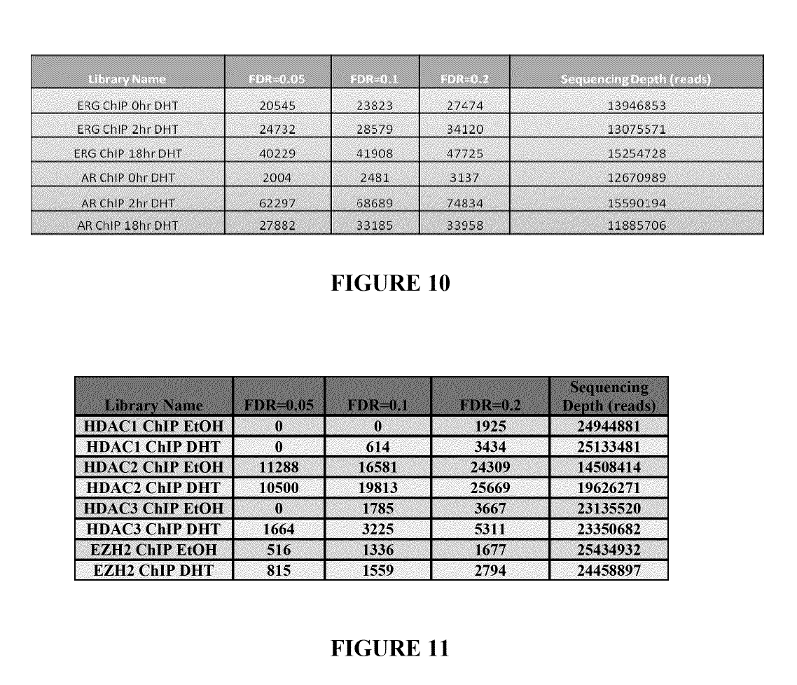

To expand our understanding of the temporal and spatial binding of AR and ERG in prostate cancer, we performed ChIP-Seq of both factors in VCaP cells at 0, 2 and 18 hrs after DHT stimulation (FIG. 10). Overall, we observed an increase in the number of AR and ERG co-localized binding events (AR+ERG) from 0 to 18 hrs, which was largely due to a sharp increase in AR binding after DHT stimulation (FIG. 2A). De novo motif analysis of ARBS and ERGBS revealed the presence of canonical ARE and ETS like motifs, respectively (FIG. 2B). The global binding profiles of the two factors, as our results at individual loci already eluded (FIG. 1C), were distinct for both factors upon DHT stimulation. In general, there was minimal AR binding in the genome prior to any stimuli (FIG. 2C-D). Most AR binding, at both AR unique and AR+ERG co-localized sites occurred 2 hrs after DHT stimulation. After 18 hrs of DHT treatment there was a global reduction in AR occupancy, an indication that at this phase of androgen signaling, the rate of AR recruitment may be outpaced by the rate of AR dissociation (FIG. 2D).

Surprisingly, in contrast to AR there was a substantial amount of ERG occupancy at both ERG unique and AR+ERG co-localized binding sites prior to DHT stimulation (FIG. 2C-D). The binding of ERG at AR+ERG co-localized binding sites was for the most part enhanced after 2 hrs DHT treatment, but not at ERG unique binding sites which suggests that at shared binding sites AR could be enhancing ERG loading (FIG. 2C-D). ERG binding at ERG unique sites eventually increased but only at the late phase of androgen signaling (18 hrs), possibly as a result of increased ERG protein expression (FIG. 1B). AR and ERG consensus motifs were found strongly enriched at AR+ERG overlap binding sites, which indicates the presence of binding motifs is one of the major determinants of AR and ERG co-occupancy.

Finally, we noticed that AR recruitment was significantly stronger at AR+ERG co-localized binding sites compared to AR unique sites (FIG. 2G), whereas ERG recruitment was the same regardless whether it was at AR+ERG co-localized binding sites or at ERG unique sites (FIG. 2H), suggesting that sites with stronger AR binding will favor ERG recruitment over their weaker counterparts.

We also examined the distribution of AR and ERG binding sites across the genome. Similar to previous observations (4), our AR ChIP-seq showed ARBS are preferentially located at distal regions that are far away from the transcriptional start sites (TSS) of genes (FIG. 2E). In contrast, ERGBS can be found at both promoter and distal sites. As for AR and ERG co-localized binding sites, they are also preferentially distributed far away from the TSS, similar to the general positioning of ARBS (FIG. 2E). In conservation analysis, AR and ERG are generally more conserved at the ChIP-Seq peak center relative to their flanking regions (FIG. 2F). ERG binding sites are the most conserved compared to AR unique and AR+ERG co-occupied binding sites (FIG. 2F), probably as a result of ERGBS being localized to the generally well conserved TSS of genes. ERG+AR overlapping binding sites, possibly owing to their higher functional importance, are more conserved than AR unique binding sites (FIG. 2F).

Taken together, our results indicate that AR and ERG binding across the genome is distinct yet share a large overlap, suggesting potential collaboration between these two factors.

Transcriptional Collaboration Between AR and ERG:

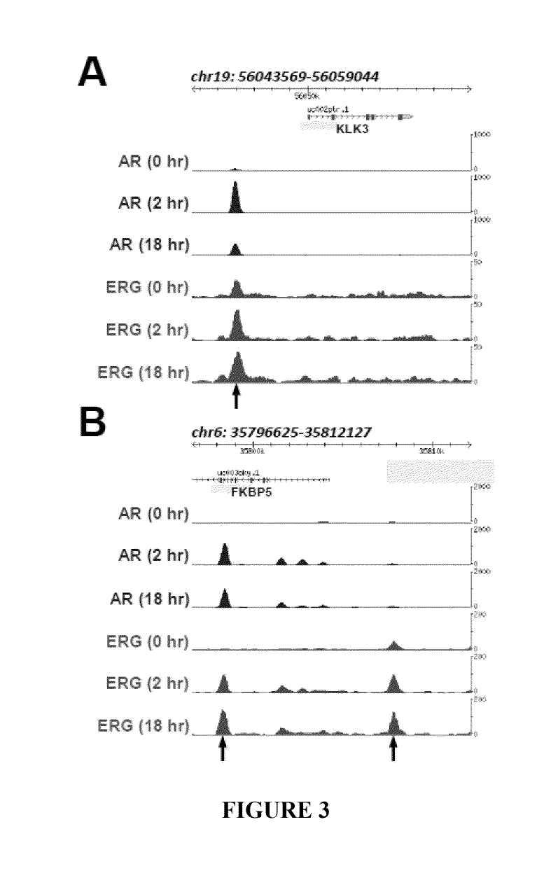

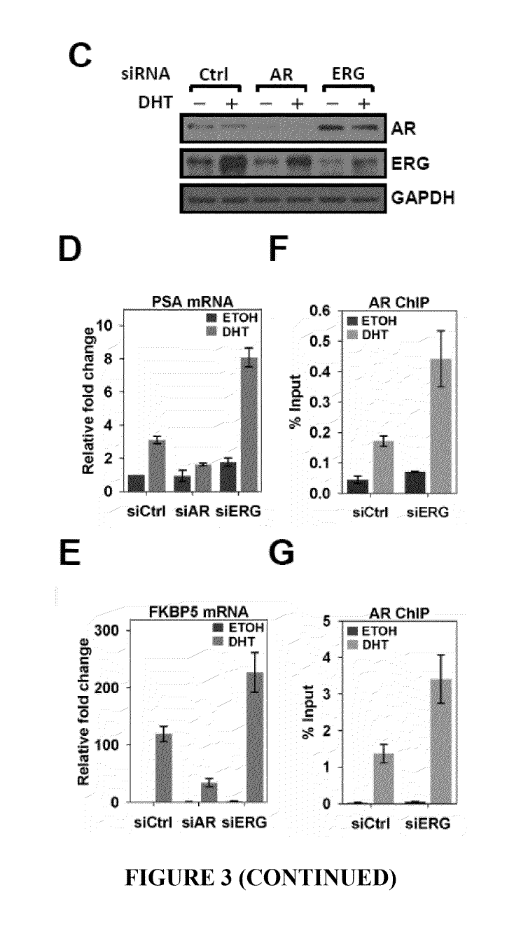

The substantial overlap of the AR and ERG cistromes suggests ERG may play a direct role in regulating AR-dependent transcription. Indeed, we noticed ERG co-localized with AR at a large number of well-known androgen-regulated genes including KLK3/PSA (FIG. 3A) and FKBP5 (FIG. 3B). To determine if ERG is required for androgen-dependent transcription, we examined the effect of siRNA-mediated knockdown of ERG on AR target gene expression. As shown in FIG. 3C-E, silencing of ERG enhanced the mRNA expression levels of both KLK3 and FKBP5 suggesting ERG functions to attenuate AR transcriptional response.

We next examined the potential mechanism underlying ERG-mediated attenuation of androgen-dependent transcription by testing the possibility that ERG might be suppressing the recruitment of AR to chromatin. To do this, we performed AR ChIP after treating VCaP cells with or without siRNA directed against ERG. As shown in FIG. 3F-G, silencing of ERG led to a significant increase in AR binding at the AR and ERG co-localized binding sites of KLK3/PSA and FKBP5.

Taken together, this result suggests the antagonistic effect of ERG on AR transcriptional activity could in part, be attributed to the reduction of AR recruitment to its cis-regulatory elements by ERG.

Integrative Transcriptional Network Between AR, ERG, and Transcriptional Corepressors in Prostate Cancer Cells:

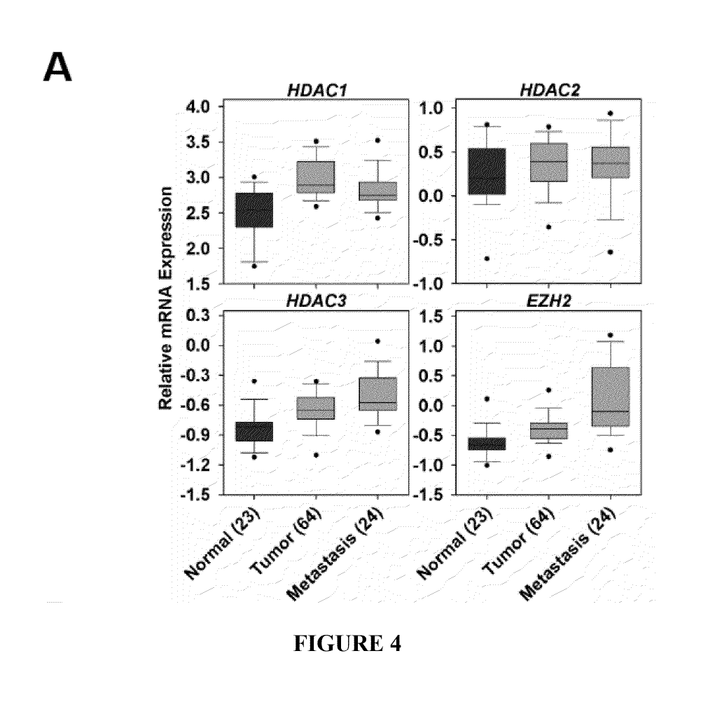

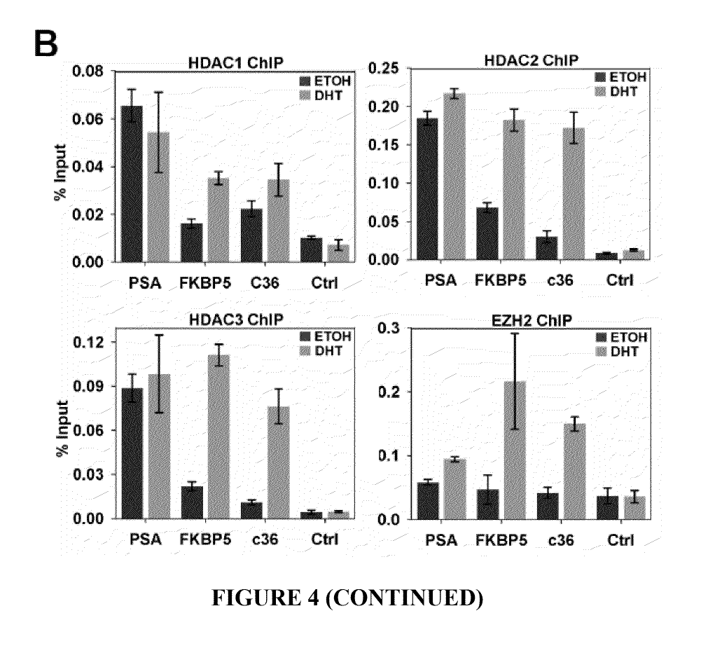

Histone deacetylases, such as HDAC1, HDAC2, and HDAC3, and the methyltransferase, EZH2, are transcriptional corepressor proteins that are commonly found over-expressed in prostate cancers (FIG. 4A) (11-12), positively correlated with ERG levels (10, 16), and play vital roles in the progression of the disease (14, 20-21). However, whether these corepressors are directly involved in regulating ERG-mediated inhibition of AR transcriptional activity has not been addressed. Previous studies have shown that HDACs can be recruited to ARBS, however this was under antiandrogen (casodex) stimulation for the repression of AR-dependent transcription (23). Although no evidence to date indicate EZH2 is directly involved in AR or NR-mediated transcription, it has however been shown to be important in regulating the activities of other transcription factors such as NFkB (24).

Based on these findings, we tested whether the attenuation of AR transcriptional activity that we observed above could be due the recruitment of one or more of these corepressors to AR and ERG co-localized binding sites. We performed ChIP assays for HDACs and EZH2 in VCaP cells before and after DHT treatment. Surprisingly, we found HDAC1, HDAC2, HDAC3, and EZH2 were all recruited to several AR and ERG co-localized binding sites that we examined including those associated with PSA and FKBP5 (FIG. 4B). Furthermore, the recruitment of these corepressors was in most cases enhanced by DHT stimulation.

Taken together, our results suggest corepressors are recruited to AR and ERG co-localized sites and may co-operate with ERG in mediating the inhibition of androgen-dependent transcription.

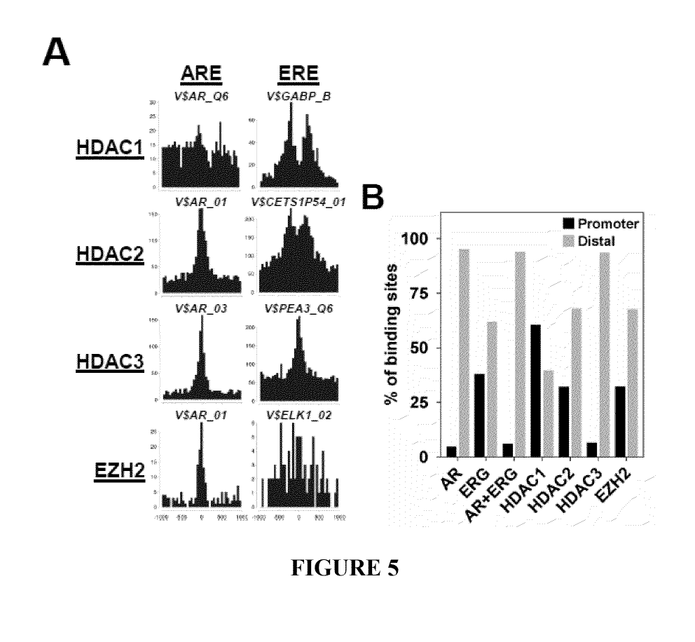

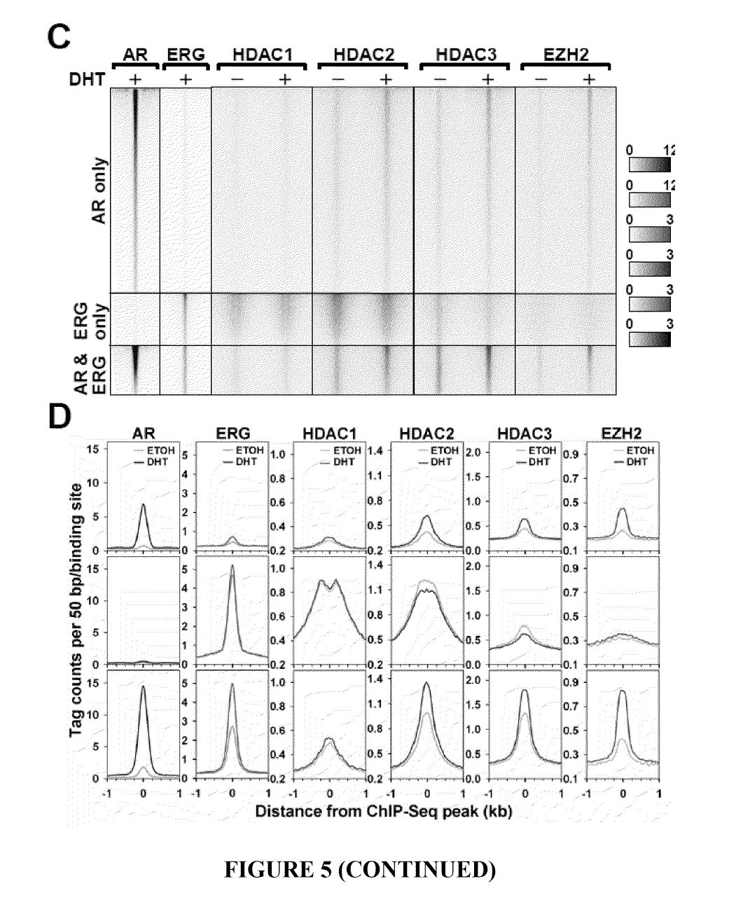

To determine the extent of co-operation between AR, ERG and the corepressors, HDACs and EZH2, we performed ChIP-Seq of these factors in VCaP cells before and after 2 hrs of DHT stimulation, the time-point corresponding to the largest overlap in AR and ERG co-localized binding (FIG. 11). We first examined the ChIP-Seq peaks of the corepressors for motif enrichment using CENTDIST (25) and found good center of distribution scores for sequences representing both AR and ERG at HDAC2, HDAC3, and EZH2 binding sites, suggesting these corepressors are indirectly recruited to chromatin via AR and ERG (FIG. 5A). In comparison, only ERG motifs were enriched for HDAC1, which indicate that HDAC1 is likely recruited mainly through ERG.

We also examined the genomic distribution of the corepressors with respect to known genes. In general, the cistrome of each individual corepressor exhibited distinct binding characteristics. For instance, HDAC1 appeared to be preferentially located at promoters, while HDAC2 and HDAC3 are predominantly found at distal enhancers (FIG. 5B). From previous binding studies of EZH2, it is generally thought that EZH2 binds mainly to promoter regions (26-29), however from our genome-wide analysis it appears that EZH2 is actually preferentially found at distal enhancers rather than at promoter regions after androgen stimulation (FIG. 5B). With respect to AR and ERG binding, we also found distinct combinations of corepressor recruitment. AR and ERG co-localized sites mainly recruited HDAC2, HDAC3, and EZH2, whereas AR unique binding sites also recruited these same factors but to a much lesser degree (FIG. 5C-D).

In contrast, ERG unique binding sites preferred to recruit HDAC 1 and -2, but not EZH2. We also noticed that HDAC2, HDAC3 and EZH2 occupancy at ARBS sites were enhanced upon androgen stimulation, with the strongest increment at AR and ERG co-occupied sites (FIG. 5D). In comparison, no changes in HDAC1 binding were observed at the same binding sites (FIG. 5D).

Taken together, our ChIP-seq results show that HDACs and EZH2 are directly integrated in the androgen signaling network and regulate AR- and ERG-dependent transcription by occupying different subsets of the AR and ERG cistrome.

HDACs and EZH2 Form a Functional Complex with ERG and AR to Attenuate Androgen-Dependent Transcription:

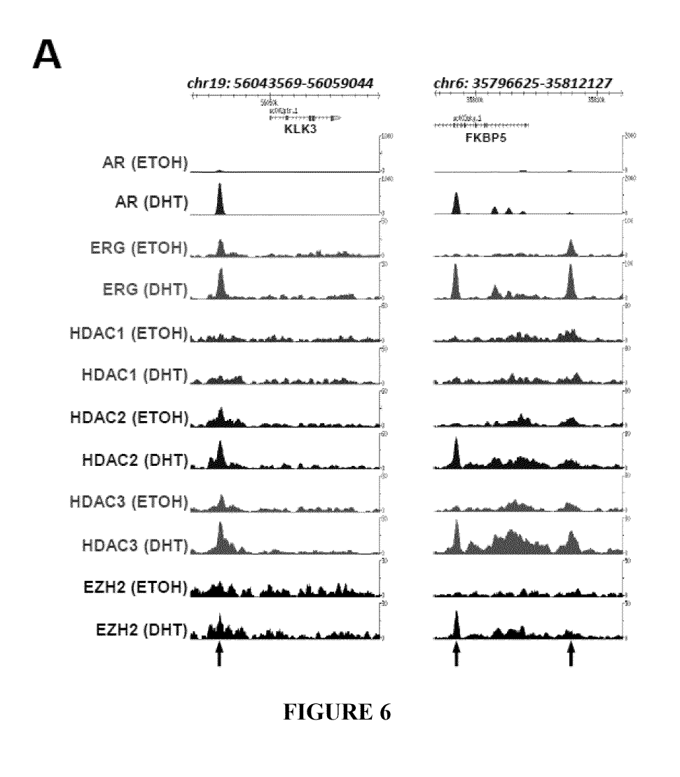

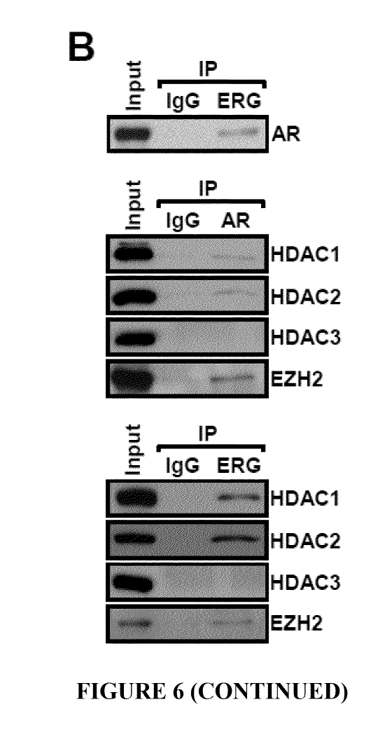

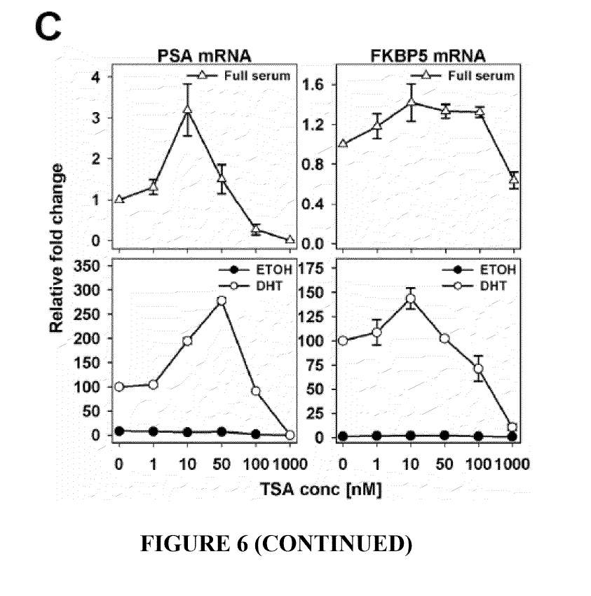

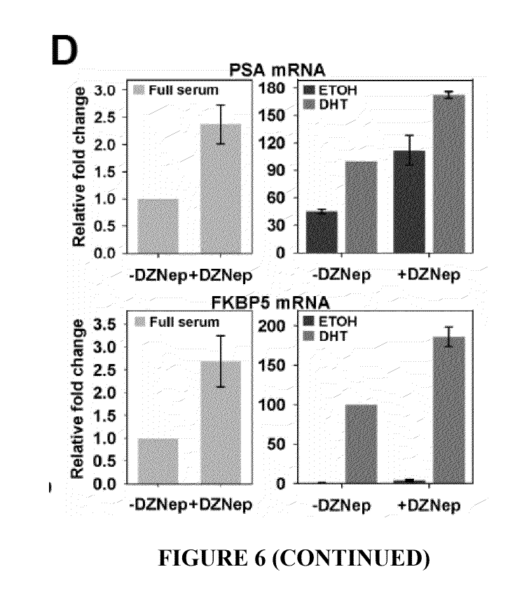

The recruitment of HDACs and EZH2 to AR and ERG co-localized binding sites across the prostate cancer genome suggests these corepressors are involved in ERG-mediated inhibition of androgen-dependent transcription. Consistent with this, we found HDACs and EZH2 recruited to AR and ERG co-localized binding sites associated with androgen direct target genes including, PSA and FKBP5 (FIG. 6A).

To determine whether HDACs and EZH2 are important in suppressing androgen-dependent transcription, we examined the transcript levels of PSA and FKBP5 after blocking the activities of the corepressors with specific small molecule inhibitors. Specifically, we used TSA (30) and DZNep (31) to inhibit the activities of HDACs and EZH2, respectively. Interestingly, we found TSA induced a biphasic transcriptional response: at low concentrations TSA enhanced PSA and FKBP5 transcript levels but at high concentrations it was repressive, suggesting a possible dual (activation and repression) function for HDACs in maintaining AR transcriptional activity (FIG. 6C). As for DZNep, it enhanced the expression of both PSA and FKBP5, indicating a role for EZH2 in suppressing AR transcriptional activity (FIG. 6D).

The recruitment of HDACs and EZH2 at AR and ERG co-localized binding sites also suggests these factors may physically interact with each other and function together as a complex. To examine this, we performed co-immunoprecipitation experiments with AR, ERG and the corepressors. Although, we had difficulty detecting interactions between HDAC3 with either AR or ERG, we were however able to observe interactions between HDAC1, HDAC2 and EZH2 with both AR and ERG (FIG. 6B).

Taken together, our results suggest HDACs and EZH2 may function together with AR and ERG as a multi-protein complex to regulate androgen-dependent transcription.

ERG, HDACs and EZH2 Mediate Prostate Cancer Progression by Inhibiting the AR-Dependent Transcription of Vinculin:

Recent studies suggest ERG inhibits differentiation, expedites EMT and promotes metastasis in prostate cancer cells by directly activating the expression of genes such as PLA1A, PLAT, PLAU, and EZH2 (10, 32). To examine if ERG inhibition of AR-dependent transcription is required for prostate cancer development, we performed a molecular concept map (MCM) analysis with androgen-upregulated genes that are associated with ERG binding sites.

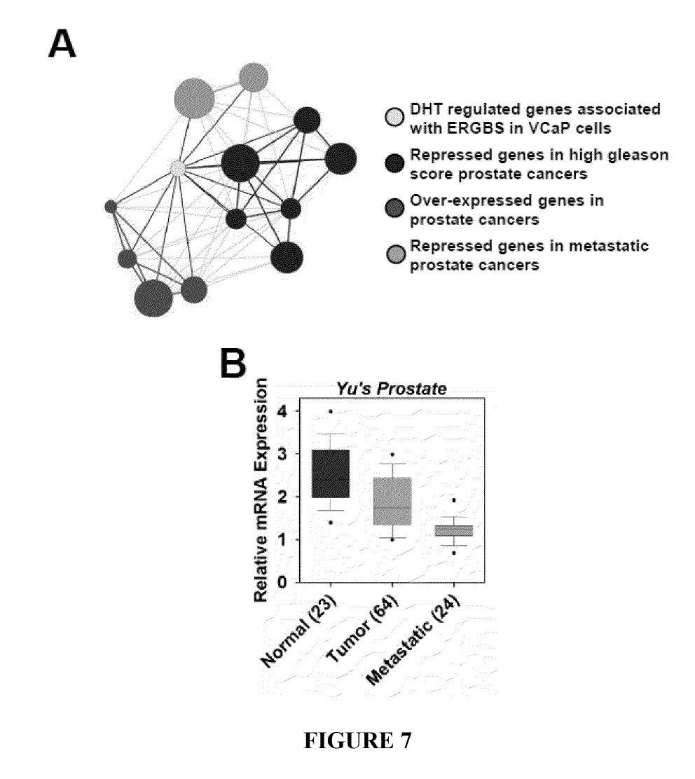

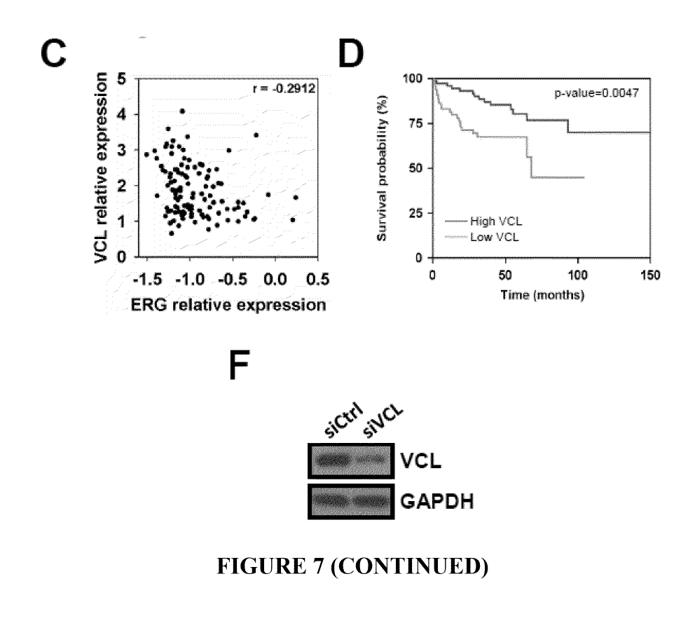

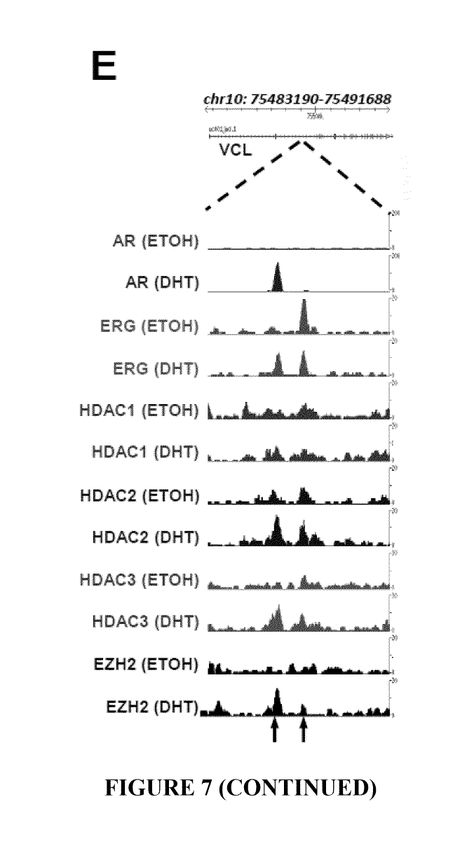

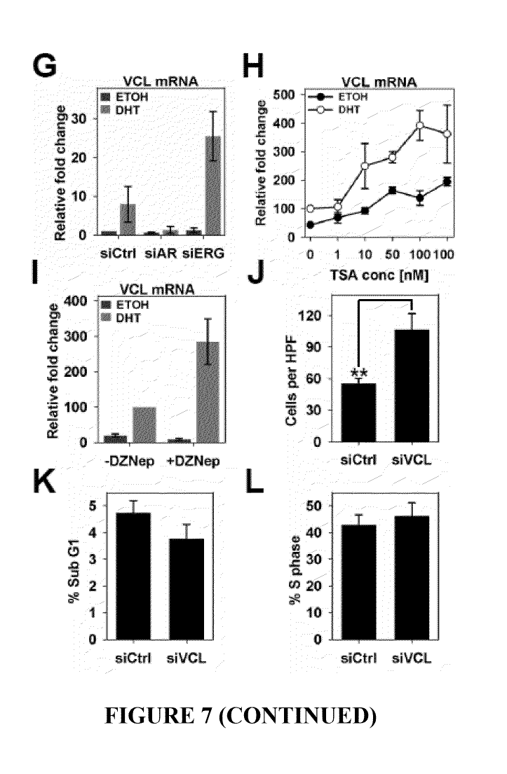

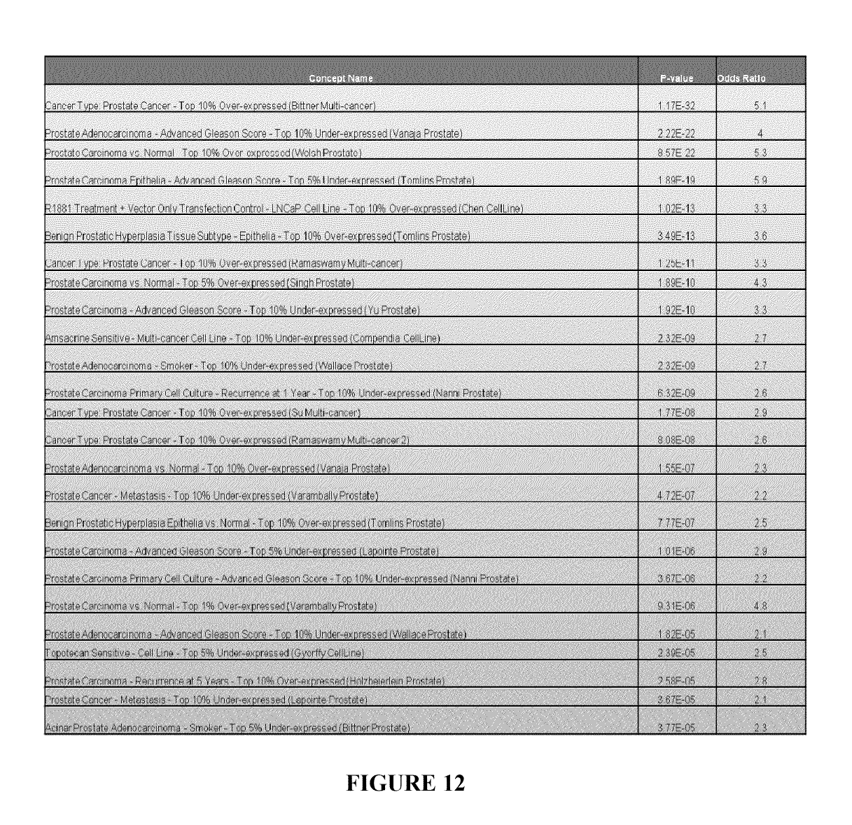

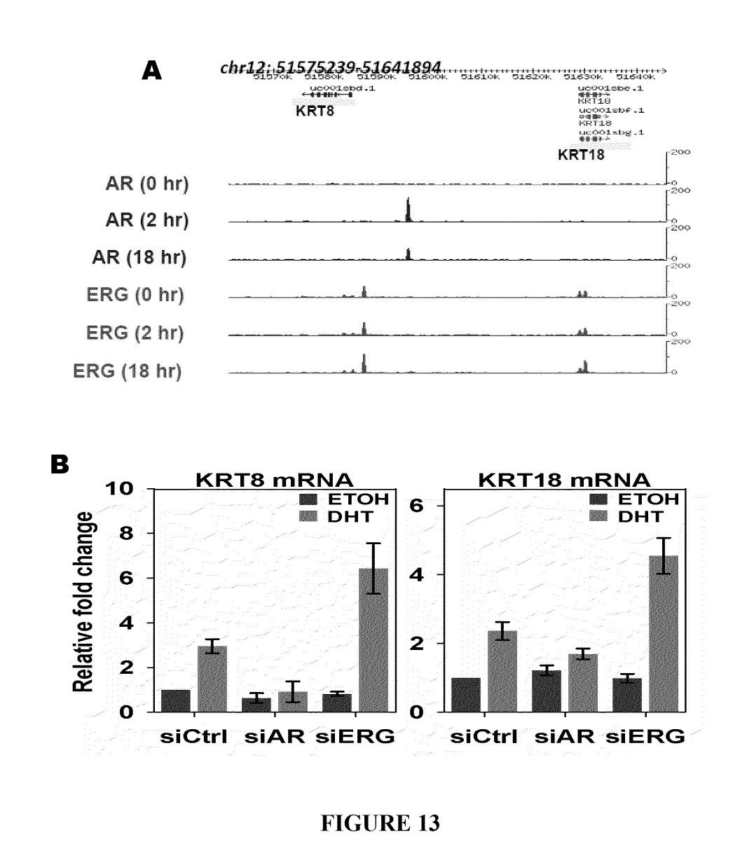

As shown in FIG. 7A and in FIG. 12, we found ERG bound androgen induced genes are enriched in several concepts related to prostate cancers, in particular with those that are over-expressed in cancers but repressed in advanced and metastatic prostate cancers. When we examined in detail the genes that are found in our defined gene set, we were able to identify previously reported mediators of MET in breast cancer including KRT8 and KRT18 (33-34) (FIG. 13). We confirmed in RT-qPCR assays the expression of these keratin genes were indeed upregulated in VCaP cells upon androgen stimulation (FIG. 13), and enhanced after ERG silencing (FIG. 13).

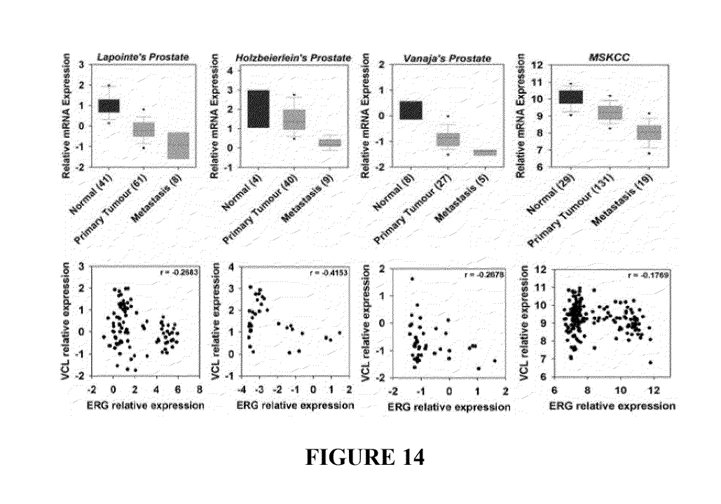

Besides keratin genes, one potential AR target gene that we speculated ERG might suppress to facilitate metastasis in prostate cancer was Vinculin (VCL). VCL is a membrane cytoskeletal protein that is required for regulating focal adhesion turnover, a process that is important for proper cell movement (35). Moreover, VCL was recently shown to interact with the MET mediator, E-Cadherin, to enhance mechanosensing (36). From clinical data in the Oncomine database, we found the mRNA expression of VCL was low in primary prostate cancers and even lower in advanced metastatic counterparts (FIG. 7B and FIG. 14). In addition, there was a negative correlation relationship between the mRNA levels of ERG and VCL, supporting our observations that ERG inhibits the androgen up-regulation of VCL expression (FIG. 7C and FIG. 14). Finally, survival analysis using data from the Taylor et al clinical study (37), we found patients with low expression of VCL have a significantly lower recurrence free survival (FIG. 7D).

To determine whether inhibition of VCL directly links ERG and AR with prostate cancer progression, we first confirmed VCL is a direct target of AR and ERG. As shown in FIG. 7E, AR and ERG are recruited to an intronic region of VCL. Moreover, siRNA mediated silencing of ERG enhanced VCL expression (FIG. 7G). From our binding and small molecule inhibitor studies, we showed ERG most likely also inhibits VCL together with HDACs and EZH2 (FIG. 7H-I). Finally, to assess if VCL is necessary for prostate cancer metastasis we performed invasion assays with VCaP cells treated with or without siRNA against VCL. Our results showed that silencing of VCL (FIG. 7F) increased the invasiveness of VCaP cells (FIG. 7J), and this was not due to differences in either cell death (FIG. 7K) or cell proliferation (FIG. 7L).

Overall, our results suggest ERG confer invasiveness to prostate cancer cells by suppressing the AR-dependent transcription of VCL.

Discussion

AR-mediated transcription is a complex multi-step process involving the coordinated recruitment of the receptor, collaborative factors, coactivators, and corepressors in a precise temporal and spatial manner. While most studies to date have focused on the role of coactivators such as SRCs and p300 in the activation of AR-dependent transcription, our understanding of how corepressors attenuate AR transcriptional activity and the functional consequences downstream of this regulation in prostate cancer, especially at the genomic level, is currently unclear. In this study, we used chromatin immunoprecipitation coupled to massively parallel sequencing (ChIP-Seq) to map the genome-wide binding profiles of AR and ERG, as well as commonly over-expressed transcriptional corepressors including HDAC1, HDAC2, HDAC3, and EZH2 in prostate cancer cells before and after androgen stimulation. Our results revealed ERG, HDACs and EZH2 are integrated into an AR transcriptional network that is required for the direct suppression of AR-dependent transcription. Moreover, we showed this AR transcription program includes genes that promote epithelial differentiation and inhibition of metastasis. Overall, our work implicates HDACs and the polycomb protein, EZH2 as novel oncogenic corepressors of androgen signaling that impede epithelial differentiation and contribute to prostate cancer progression, in part through modulating the transcriptional output of AR and ERG.

TMPRSS2:ERG is the most common form of ETS gene fusion found in prostate cancers (6). Previous studies showed that ERG can bind to the enhancer ARBS of the androgen-regulated gene, PSA, suggesting a potential collaboration between AR and ERG (9). From our global analysis of these two factors, we found there is a widespread co-localization of AR and ERG after 2 hrs of DHT stimulation (FIG. 2), which indicates the collaboration between these two factors occurs throughout the prostate cancer genome. During the course of this work, a genome-wide map of ERG, also in VCaP cells, was reported (10). Although the experimental conditions are different between the two studies, the general observation of extensive overlap between AR and ERG binding was similar. However, in contrast to the other study, which examined ERG binding only under full serum condition, our study here provides an extensive profile of AR and ERG binding before and after androgen stimulation at short and long time intervals. From our time-course ChIP-Seq of AR and ERG, we uncovered several surprising insights into the mechanism of AR- and ERG-mediated transcriptional regulation during androgen signaling. Specifically, our study revealed that ERG in general was pre-bound to chromatin at ERG unique and AR+ERG co-localized binding sites prior to androgen stimulation (FIG. 2). This finding was unexpected since the expression of ERG is thought to be androgen-regulated due to a fusion event (5). However, as shown in our western blot analysis of ERG (FIG. 1B), there is already a significant level of the protein expressed in VCaP cells before androgen stimulation, which likely explains why ERG can bind to chromatin before androgen stimulation. Although ERG is pre-bound to chromatin, our time-course ChIP-Seq also showed that the recruitment of ERG to AR+ERG co-localized binding sites can be further enhanced with short-term DHT stimulation, while the increment of ERG at ERG unique sites occurred mainly after a rise in ERG levels stemming from prolonged androgen stimulation (FIG. 2). This observation suggests that additional ERG recruitment to ARBS may be enhanced by AR binding. Moreover, this result shows that ERG is unlike other transcriptional repressors of nuclear receptor such as NKX3-1 and LEF-1, which compete with the Estrogen Receptor (ER) for binding to the ER binding sites (38). Finally, we also noticed from our ChIP-Seq study that apart from being recruited at AR-bound enhancers, ERG was frequently located at the promoters of AR target genes as well (data not shown). This binding was usually independent of AR recruitment. Whether ERG binding at the promoter region is required for the repression of AR target genes will require further studies in the future.

Besides AR and ERG, numerous transcriptional corepressors are also frequently over-expressed in prostate cancers (11-12). Through genome-wide binding analysis, we discovered the existence of a closely knitted and intricate transcriptional network between AR and ERG, as well as the frequently over-expressed corepressor proteins, HDAC1, HDAC2, HDAC3, and EZH2.

Strikingly, this transcriptional network is highly regulated by and responsive to androgen stimulation (FIG. 5). In general, our study showed that androgen signaling culminates in an increased occupancy of AR, ERG, HDAC2, HDAC3, and EZH2 to shared elements in the network (FIG. 5). Although HDACs have been shown to play a role in nuclear receptor transcription (23, 39), our finding that EZH2 is co-localized at ARBS was rather surprising since EZH2 is mainly associated with the methylation of histones at the promoter of repressed genes (26-29). Even though our results suggest that ERG can engage HDACs and EZH2 (FIG. 6B), the mechanism of their transcriptional co-operation still elucidates us. For example, we hypothesized that recruitment of ERG to the regulatory elements would lead to enhanced HDACs and EZH2 occupancy. Since HDAC2 has the strongest co-localization with ERG, we examined the effect of HDAC2 recruitment upon ERG knockdown. Our results revealed no significant change in the strength of HDAC2 binding at several AR and ERG co-occupied sites after ERG knockdown (data not shown). This indicates that ERG may not be the only factor responsible for the recruitment of HDAC2. In view of this, it would be important to determine the nature of the corepressor complex in the absence of ERG in further studies.

Since TMPRSS2:ERG is a recurrent gene fusion widely expressed in prostate cancers, it was not surprising to find it plays vital roles in prostate cancer initiation and progression (40-41). Although our work and others (9-10) showed that ERG repressed AR-mediated induction of differentiation markers, these markers have no major known functional role in prostate cancer differentiation and progression (42). However, from our gene association analysis, we found that ERG-associated androgen induced genes are highly associated with metastatic prostate cancers and thus might be involved in cellular processes such as Epithelial Mesenchymal Transition (EMT) that promote cell invasion.

In the EMT process, epithelial markers including keratins and E-Cadherins will be replaced with mesenchymal markers such as Vimentin and N-Cadherins (43). This change in the composition of the cell adhesion and cytoskeleton molecules will inevitably lead to a decrease in cell adhesion as well as cell-cell cohesion and in turn, culminate into an increase in cell invasiveness (43). Interestingly, from our work we noticed several epithelial keratin proteins (e.g. KRT8 and KRT18) were upregulated by androgen stimulation (FIG. 14). Thus, our findings suggest that by repressing the expression of epithelial cell adhesion and cytoskeletal molecules through inhibition of AR signaling, ERG could potentially promote EMT and confer metastatic properties. Indeed, we identified the AR-induced but ERG repressed gene, VCL, as a novel suppressor of prostate invasiveness. Interestingly, VCL was recently shown to potentiate E-cadherin mechanosensing (36) and therefore may be required for the optimal function of E-cadherin.

Given that androgen is the main driver of prostate cancer progression, it was contradicting that studies showed high doses of DHT treatment could slow prostate cancer progression (44-45). This phenomenon could be partially explained by the dual opposing role of AR in prostate cancer progression. On one hand, AR can promote proliferation and inhibits apoptosis. On the other hand, AR can also halt cancer progression and metastasis by inducing differentiation and enhancing an epithelial phenotype. Moreover, a recent study by Zhu and Kyprianou (46) showed that a low AR content is required for an EMT phenotype in prostate cancers. These findings suggest that AR signaling requires fine-tuning to an optimal level in order to favor prostate cancer progression. Our study is in agreement with these observations and provides a molecular explanation for how AR can regulate these two distinct processes in prostate cancer. Taken together, our findings show that a highly integrated transcriptional network of AR and ERG, together with HDACs and EZH2, exists to advance the development of prostate cancers to a metastable state by restraining epithelial differentiation and promoting EMT through regulated suppression of AR signaling.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. As used in this specification and the appended claims, all ranges or lists as given are intended to convey any intermediate value or range or any sublist contained therein. Unless defined otherwise all technical and scientific terms used herein have the same meaning as commonly understood to one of ordinary skill in the art to which this invention belongs.

Concentrations given in this specification, when given in terms of percentages, include weight/weight (w/w), weight/volume (w/v) and volume/volume (v/v) percentages.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

1. Heinlein C A & Chang C (2004) Androgen receptor in prostate cancer. Endocrine reviews 25(2):276-308.
2. Heinlein C A & Chang C (2002) Androgen receptor (AR) coregulators: an overview. Endocrine reviews 23(2):175-200.
3. Gregory C W, et al. (2001) A mechanism for androgen receptor-mediated prostate cancer recurrence after androgen deprivation therapy. (Translated from eng) Cancer Res 61(11):4315-4319 (in eng).
4. Wang Q, et al. (2007) A hierarchical network of transcription factors governs androgen receptor-dependent prostate cancer growth. Molecular cell 27(3):380-392.
5. Tomlins S A, et al. (2005) Recurrent fusion of TMPRSS2 and ETS transcription factor genes in prostate cancer. Science (New York, N.Y.) 310(5748):644-648.
6. Kumar-Sinha C, Tomlins S A, & Chinnaiyan A M (2008) Recurrent gene fusions in prostate cancer. Nature reviews. Cancer 8(7):497-511.
7. Massie C E, et al. (2007) New androgen receptor genomic targets show an interaction with the ETS1 transcription factor. EMBO reports 8(9):871-878.
8. Shin S, et al. (2009) Induction of prostatic intraepithelial neoplasia and modulation of androgen receptor by ETS variant 1/ETS-related protein 81. Cancer Res 69(20):8102-8110.
9. Sun C, et al. (2008) TMPRSS2-ERG fusion, a common genomic alteration in prostate cancer activates C-MYC and abrogates prostate epithelial differentiation. Oncogene 27(40):5348-5353.
10. Yu J, et al. (2010) An integrated network of androgen receptor, polycomb, and TMPRSS2-ERG gene fusions in prostate cancer progression. Cancer cell 17(5):443-454.
11. Weichert W, et al. (2008) Histone deacetylases 1, 2 and 3 are highly expressed in prostate cancer and HDAC2 expression is associated with shorter PSA relapse time after radical prostatectomy. Br J Cancer 98(3):604-610.
12. Varambally S, et al. (2002) The polycomb group protein EZH2 is involved in progression of prostate cancer. Nature 419(6907):624-629.
13. Glozak M A & Seto E (2007) Histone deacetylases and cancer. (Translated from eng) Oncogene 26(37):5420-5432 (in eng).
14. Wang L, et al. (2009) Increased expression of histone deacetylaces (HDACs) and inhibition of prostate cancer growth and invasion by HDAC inhibitor SAHA. Am J Transl Res 1(1):62-71.
15. Gupta S, et al. (2010) FZD4 as a mediator of ERG oncogene-induced WNT signaling and epithelial-to-mesenchymal transition in human prostate cancer cells. Cancer Res 70(17):6735-6745.
16. Iljin K, et al. (2006) TMPRSS2 fusions with oncogenic ETS factors in prostate cancer involve unbalanced genomic rearrangements and are associated with HDAC 1 and epigenetic reprogramming. Cancer Res 66(21):10242-10246.
17. Bjorkman M, et al. (2008) Defining the molecular action of HDAC inhibitors and synergism with androgen deprivation in ERG-positive prostate cancer. Int J Cancer 123(12):2774-2781.
18. Cao R, et al. (2002) Role of histone H3 lysine 27 methylation in Polycomb-group silencing. (Translated from eng) Science 298(5595):1039-1043 (in eng).
19. Cao R & Zhang Y (2004) The functions of E(Z)/EZH2-mediated methylation of lysine 27 in histone H3. (Translated from eng) Curr Opin Genet Dev 14(2):155-164 (in eng).
20. Yu J, et al. (2010) The neuronal repellent SLIT2 is a target for repression by EZH2 in prostate cancer. Oncogene 29(39):5370-5380.
21. Yu J, et al. (2007) A polycomb repression signature in metastatic prostate cancer predicts cancer outcome. Cancer Res 67(22):10657-10663.
22. Cao Q, et al. (2008) Repression of E-cadherin by the polycomb group protein EZH2 in cancer. Oncogene 27(58):7274-7284.
23. Shang Y, Myers M, & Brown M (2002) Formation of the androgen receptor transcription complex. (Translated from eng) Mol Cell 9(3):601-610 (in eng).
24. Lee S T, et al. (2011) Context-Specific Regulation of NF-kappaB Target Gene Expression by EZH2 in Breast Cancers. (Translated from eng) Mol Cell 43(5):798-810 (in eng).
25. Mang Z, Chang C W, Goh W L, Sung W K, & Cheung E (2011) CENTDIST: discovery of co-associated factors by motif distribution. Nucleic Acids Res.
26. Yu J, et al. (2007) Integrative genomics analysis reveals silencing of beta-adrenergic signaling by polycomb in prostate cancer. (Translated from eng) Cancer Cell 12(5):419-431 (in eng).
27. Fujii S, Ito K, Ito Y, & Ochiai A (2008) Enhancer of zeste homologue 2 (EZH2) down-regulates RUNX3 by increasing histone H3 methylation. (Translated from eng) J Biol Chem 283(25):17324-17332 (in eng).
28. Ku M, et al. (2008) Genomewide analysis of PRC1 and PRC2 occupancy identifies two classes of bivalent domains. (Translated from eng) PLoS Genet 4(10):e1000242 (in eng).
29. Margueron R, et al. (2008) Ezh1 and Ezh2 maintain repressive chromatin through different mechanisms. (Translated from eng) Mol Cell 32(4):503-518 (in eng).
30. Codd R, Braich N, Liu J, Soe C Z, & Pakchung A A (2009) Zn(II)-dependent histone deacetylase inhibitors: suberoylanilide hydroxamic acid and trichostatin A. (Translated from eng) Intl Biochem Cell Biol 41(4):736-739 (in eng).
31. Tan J, et al. (2007) Pharmacologic disruption of Polycomb-repressive complex 2-mediated gene repression selectively induces apoptosis in cancer cells. (Translated from eng) Genes Dev 21(9):1050-1063 (in eng).
32. Tomlins S A, et al. (2008) Role of the TMPRSS2-ERG gene fusion in prostate cancer. Neoplasia (New York, N.Y.) 10(2):177-188.
33. Buhler H & Schaller G (2005) Transfection of keratin 18 gene in human breast cancer cells causes induction of adhesion proteins and dramatic regression of malignancy in vitro and in vivo. (Translated from eng) Mol Cancer Res 3(7):365-371 (in eng).
34. Tomaskovic-Crook E, Thompson E W, & Thiery J P (2009) Epithelial to mesenchymal transition and breast cancer. (Translated from eng) Breast Cancer Res 11(6):213 (in eng).
35. Saunders R M, et al. (2006) Role of vinculin in regulating focal adhesion turnover. Eur J Cell Biol 85(6):487-500.
36. le Duc Q, et al. (2010) Vinculin potentiates E-cadherin mechanosensing and is recruited to actin-anchored sites within adherens junctions in a myosin II-dependent manner. (Translated from eng) J Cell Biol 189(7):1107-1115 (in eng).
37. Taylor B S, et al. (2010) Integrative genomic profiling of human prostate cancer. (Translated from eng) Cancer Cell 18(1):11-22 (in eng).
38. Holmes K A, Song J S, Liu X S, Brown M, & Carroll J S (2008) Nkx3-1 and LEF-1 function as transcriptional inhibitors of estrogen receptor activity. Cancer research 68(18):7380-7385.
39. Burd C J, Morey L M, & Knudsen K E (2006) Androgen receptor corepressors and prostate cancer. (Translated from eng) Endocr Relat Cancer 13(4):979-994 (in eng).
40. Zong Y, et al. (2009) ETS family transcription factors collaborate with alternative signaling pathways to induce carcinoma from adult murine prostate cells. Proceedings of the National Academy of Sciences of the United States of America 106(30): 12465-12470.
41. King J C, et al. (2009) Cooperativity of TMPRSS2-ERG with PI3-kinase pathway activation in prostate oncogenesis. Nature genetics 41(5):524-526.
42. Chen Y & Sawyers C L (2010) Coordinate transcriptional regulation by ERG and androgen receptor in fusion-positive prostate cancers. (Translated from eng) Cancer Cell 17(5):415-416 (in eng).
43. Lee J M, Dedhar S, Kalluri R, & Thompson E W (2006) The epithelial-mesenchymal transition: new insights in signaling, development, and disease. The Journal of cell biology 172(7):973-981.
44. Tsihlias J, et al. (2000) Involvement of p27Kip1 in G1 arrest by high dose 5 alpha-dihydrotestosterone in LNCaP human prostate cancer cells. (Translated from eng) Oncogene 19(5):670-679 (in eng).
45. Hofman K, Swinnen J V, Verhoeven G, & Heyns W (2001) E2F activity is biphasically regulated by androgens in LNCaP cells. Biochemical and biophysical research communications 283(1):97-101.
46. Thu M-L & Kyprianou N (2010) Role of androgens and the androgen receptor in epithelial-mesenchymal transition and invasion of prostate cancer cells. The FASEB journal: official publication of the Federation of American Societies for Experimental Biology 24(3):769-777.
47. Tan S K, et al. (2011) AP-2gamma regulates oestrogen receptor-mediated long-range chromatin interaction and gene transcription. EMBO J.
48. Xu H, et al. (2010) A signal-noise model for significance analysis of ChIP-seq with negative control. (Translated from eng) Bioinfonnatics 26(9):1199-1204 (in eng).
49. Yu Y P, et al. (2004) Gene expression alterations in prostate cancer predicting tumor aggression and preceding development of malignancy. (Translated from eng) J Clin Oncol 22(14):2790-2799 (in eng).

What is claimed is:

1. A method of modulating a prostate cancer cell, the method comprising administering to the prostate cancer cell an agent that augments vinculin expression levels in the cell.

2. The method of claim 1, wherein the prostate cancer cell has been identified as having low expression levels of vinculin as compared with a non-cancerous prostate cell.

3. The method of claim 1, wherein the agent is an expression vector encoding vinculin.

4. The method of claim 4, wherein the expression vector encodes a vinculin protein comprising a sequence of any one of SEQ ID NOs.: 1 to 4.

5. The method of claim 1, wherein the agent is an agent that inhibits the activity of one or more of ERG, EZH2, HDAC1, HDAC2 and HDAC3.

6. The method of claim 5, wherein the agent is a chemical inhibitor.

7. The method of claim 6 wherein the agent comprises multiple agents, each of said multiple agents inhibiting one of ERG, EZH2, HDAC1, HDAC2 and HDAC3.

8. The method of claim 5, wherein the agent is an siRNA, an antisense RNA molecule or a DNA enzyme.

9. The method of claim 8 wherein the agent comprises multiple agents, each of said multiple agents inhibiting one of ERG, EZH2, HDAC1, HDAC2 and HDAC3.

10. The method of claim 1, wherein the prostate cancer cell is an in vitro cell.

11. The method of claim 1, wherein the prostate cancer cell is an in vivo cell.

12. A method of diagnosing prostate cancer disease state in a subject, the method comprising detecting vinculin levels in a sample of prostate cells obtained from a subject and comparing the levels with vinculin levels in a non-cancerous prostate cell, wherein vinculin expression levels that are lower in the sample obtained from the subject as compared to vinculin expression levels in a non-cancerous prostate cell are indicative of prostate cancer.

13. The method of claim 12, wherein the lower vinculin expression levels are indicative that the prostate cancer cell from the subject has a predisposition to be an invasive cancer.
